SEQUENCE LISTING (15 sequences)

SEQ ID NO: 1 - protein, 1273 aa, Unknown
  Coronavirinae
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Unknown
  Coronavirinae
atgtttgtattcttggtacttctcccattggtatcttctcaatgcgttaaccttaccacacgcacccaactgcccccggcctacactaatagctttacgcggggtgtctactatcccgacaaagtctttcgatccagtgtgctccactccacccaggatcttttccttccctttttttctaatgttacgtggttccacgcaatccatgtatccggtacgaatgggacaaaacgctttgacaatccagtgctgccatttaatgatggagtgtactttgcatctaccgagaagagtaacatcatcagaggatggatcttcggaacgaccttggactccaaaacgcaatccttgcttatcgttaacaatgcaacgaatgttgtcatcaaagtttgcgaattccaattctgtaacgatcccttcctcggtgtttattatcataaaaataataaatcttggatggaaagtgagttccgcgtatacagttccgccaataattgtaccttcgaatacgtaagtcaaccgttcttgatggatctggaaggtaaacagggtaactttaagaaccttcgggagtttgtttttaagaacatagacggctactttaagatctatagtaaacatacgccaattaacttggttagagatctcccgcaggggttttcagcattggagccgctcgtcgacctccccataggtataaatataactcggtttcaaacactgctggcgctccaccgcagctacctgacgcctggggattcttcttccggttggactgcaggcgctgctgcatattatgtagggtacctgcaaccgagaacctttctccttaagtacaacgagaatggcactattacggacgctgtcgattgtgcactcgaccccttgagtgagacgaagtgtacactgaaaagctttactgttgaaaagggaatatatcagacatccaactttagagttcagccaacagaatccatcgttcgatttcccaatattacaaatctctgtccgttcggagaggtctttaatgctacccgattcgcgtcagtatacgcctggaacagaaagagaatttctaactgtgttgcagattatagtgtcctgtataattctgcgtcttttagcacttttaagtgctacggcgttagccccactaagttgaacgacctttgtttcactaacgtgtatgccgactcattcgtcataagaggcgacgaagttagacaaattgcaccgggccagacgggaaagattgcggactacaactataaattgcctgacgactttacaggatgtgtcatcgcctggaatagtaataaccttgactccaaagtcggtggcaattacaattacttgtaccggctgttcaggaagtctaatctcaaaccttttgagcgagatatcagcacggaaatttatcaagctggtagcactccatgtaacggggttgagggttttaattgttattttccattgcaatcatatggattccaaccgactaacggtgttgggtatcaaccatacagagtggtggttttgtcatttgaacttctgcatgcccctgcaacagtgtgcggaccgaagaagagtacgaaccttgtaaagaacaagtgcgtcaacttcaactttaatggtctgacgggtaccggcgttctgacggaatccaataaaaagttcttgccctttcagcagttcgggcgagatatcgccgacactactgatgcggtgcgagatcctcagacacttgagatcctcgatattaccccatgtagttttggtggtgtgtctgtgattacacccggcaccaatacgtcaaatcaggtcgcagtcttgtaccaagacgtgaactgcaccgaagttcctgtagccattcacgctgatcaattgacaccgacatggagggtgtactccaccggatctaacgtgttccagacccgcgcggggtgtcttatcggcgcagaacatgtgaacaactcttacgaatgtgatattcctatcggtgcaggcatctgtgcctcataccagacacaaacgaactcaccaaggagggcaaggtcagtagcctcacaaagcataatagcctatacgatgagtcttggtgcggagaactctgtggcgtactctaataactctatcgccataccgactaacttcaccatttctgttacgaccgagatcctcccagtttccatgactaagacaagtgtggattgtacaatgtacatctgcggcgacagtactgagtgcagtaacctgcttctgcagtacgggtccttctgcacacaacttaaccgggcgctgactggtatagcggttgaacaagacaagaacactcaagaggtcttcgcacaagtaaaacaaatatacaaaacaccacctattaaagatttcggcgggtttaattttagccaaatccttccagaccccagcaaaccctctaagcgcagcttcattgaggatctgctgtttaacaaggtcaccctggcagacgcgggctttatcaagcaatacggtgactgcctgggggatatcgcggctcgagaccttatatgtgcgcaaaaatttaatggacttaccgtacttcctccattgctgactgacgagatgatagcacagtatacatctgcactgctcgccggtacaattacatcagggtggacatttggggcgggagctgcgctccagataccgttcgcgatgcagatggcgtataggtttaatggaattggtgtcacgcaaaacgttctctatgaaaaccagaagctgatagcaaatcagttcaattccgcgattggtaagatacaagattcattgtctagtacggcctctgcactcggaaaactccaagatgtagtgaaccaaaacgcccaagccctgaatacactcgtaaaacagctctctagtaattttggggccatttcctccgtattgaacgacatcttgagtcgcttggataaggtagaagcagaagtacaaattgaccggttgatcacgggcagacttcaatcacttcagacttatgttactcagcagcttatacgagctgcagaaattcgcgcctctgcgaacctggccgccactaaaatgtcagaatgtgtactgggacagagcaaacgggtggatttctgcggaaagggctatcatctgatgagttttccccagtctgcgcctcatggtgtagtatttcttcatgtcacatatgtaccagcccaagaaaaaaatttcacaacggcgcccgcgatttgccatgacggtaaggcgcattttcctcgcgagggcgttttcgtgtctaacggtactcactggttcgtaacacagcgaaacttttacgagcctcagataatcacgacggataacacatttgtctccggcaactgcgatgtggtcatcggtatagtgaacaatacggtatatgatccgctgcagccagagctcgacagtttcaaggaggagcttgacaaatactttaagaaccatacctccccagacgtagacctcggagacatatctggtatcaatgcctccgtggttaacatacaaaaggagatagatagactgaatgaggtggcgaagaatctgaatgagtctctcatagatctgcaggaactcggtaaatatgaacaatacatcaagtggccttggtacatctggctggggttcatagcgggcctgatcgcgatcgtgatggtaactataatgttgtgttgcatgacctcctgctgctcatgccttaaaggttgttgttcttgcgggagctgctgcaagttcgatgaggatgattcagaacccgtcttgaagggcgtaaaacttcactatacgtaa

SEQ ID NO: 3 - protein, 1250 aa, Unknown
  Coronavirinae
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 4 - protein, 1250 aa, Unknown
  Coronavirinae
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 5 - protein, 1252 aa, Unknown
  Coronavirinae
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 6 - DNA, 3750 bp, Unknown
  Coronavirinae
atgttcgtcttcctggtgctgctgcctttggtttcatcccaatgcgtgaacctcaccacacgtacacagctgccgccggcctatacgaactcatttaccagaggcgtctactatccggataaggtctttcggagtagcgtcctgcacagcacccaggatctgtttctgcccttcttctcaaacgtgacatggtttcacgcgatctcaggcactaacgggacgaaacgattcgacaatccggtgctccccttcaacgacggcgtgtacttcgcatcaaccgaaaagtccaatatcattcgtggttggatcttcgggaccacacttgacagtaaaactcaatctctgttgatcgtgaacaatgccaccaacgtggtaatcaaggtgtgcgagttccagttctgcaacgacccgttcctgggcgtgcataagaataataagtcatggatggagagcgaatttcgggtgtactcatccgctaacaactgtaccttcgagtacgtttcacaaccgttcctcatggatcttgagggcaagcaaggcaactttaagaacctgcgggagttcgtattcaagaacatcgacggatacttcaaaatctactcaaaacatacccccataaacctcgtaagggacctgccacaaggcttctctgcgctggagcccctcgtggacctgcctatcggcatcaatattacccggttccagacactgttggccctgcaccggtcttatctcaccccgggagacagctcatctggctggacggccggggcagcagcatactatgtcgggtacctgcagccacgcacctttctgctcaagtacaacgagaacgggacaatcaccgacgcagtggattgcgccctggacccactgtccgagactaaatgcaccctcaagagctttaccgtcgagaagggtatataccagacaagcaatttccgcgtgcagcctactgagagtatcgtgcgcttcccaaacatcactaatctgtgtccattcggagaggtattcaatgcaacaaggttcgccagtgtgtacgcgtggaatcggaaacgaattagtaattgcgtggcagactacagcgttctttacaacagcgctagtttctcaaccttcaaatgctacggggtttccccgacgaagttgaacgacctgtgtttcacaaacgtgtacgctgacagtttcgtcatccggggtgacgaggttaggcagatagcccccggacagaccgggaaaatcgccgactacaactacaagctccctgacgacttcacgggttgtgtaatcgcctggaattctaataacctggacagcaaagtgggcggaaactacaactatctttaccgcctgttccgtaaaagcaacctgaagccattcgaacgcgacatatctacggagatataccaagctggctctaccccatgcaacggagtggaagggttcaactgctatttccccctgcagagctacgggtttcagccaacctacggcgtcggatatcagccctatagggtcgttgtcctgtccttcgagctgctgcacgccccggcgaccgtgtgcggcccaaagaaatcaacgaacctggtgaagaataagtgcgttaactttaattttaacggcctcactgggactggggtcttgacagaatccaataagaaattccttccctttcagcagttcggtcgcgatatcgacgatacaacagacgccgttcgggacccccaaactttggaaatactcgatatcactccctgcagtttcggcggcgtgtccgtcatcacgcctggcaccaacaccagcaatcaagtagccgtcttgtaccaaggcgtcaattgtaccgaggtgccagtcgcaatccacgccgaccagctcaccccaacgtggagagtgtactcaacgggcagcaacgtattccagaccagggcgggttgcctgatcggcgccgagcacgtgaataatagctacgaatgcgatatcccaatcggggcgggcatttgtgcctcataccaaacccaaacaaactcacacaggcgggctaggtctgttgccagccagagcataatcgcttataccatgtccctgggcgctgagaacagtgtggcctatagcaacaattccatagctattcctatcaacttcacaatctccgtaactactgagatccttcctgtctccatgactaaaacttctgtcgattgcaccatgtatatctgcggcgacagcacagagtgttcaaacctgctgctgcagtacggttccttctgcacccagctgaatagggcgttgaccggtatcgcggtcgagcaggataagaatacgcaggaagtgttcgcccaggttaagcagatttataagacgccccctatcaaggacttcggcggattcaacttctcccagattctgcctgaccccagcaagccttctaaaagatccttcatcgaagatctattgtttaataaggtaactttggccgacgccgggtttattaagcagtacggcgactgtttgggcgacatcgcagcaagggatctgatctgcgcccagaaattcaatggactgacagtgctgcccccactcttgacggacgagatgatcgcccagtataccagcgcgctcctggccggcaccattaccagtggatggacattcggcgctggcgcggctctgcagatccctttcgcaatgcagatggcgtaccgtttcaacggcataggtgtaacccaaaacgtactgtacgaaaatcagaagctgatagctaatcagttcaacagcgccatcgggaagatccaggattctctgtcaagcactgcctcagccctcgggaagttgcaggacgtagttaatcagaacgctcaggccttgaataccctcgtaaagcagttgtccagtaacttcggagctatctctagcgtgttgaacgacatattggccaggctcgataaggtagaagccgaggtacagatcgaccgactgattaccggacgactgcagtcccttcaaacttacgtaacgcagcagctgatcagggccgccgagattagggccagcgcgaacctcgccgcaaccaagatgagtgaatgcgttctgggccagagtaagcgtgtggacttctgcggcaaaggttaccacctcatgtcttttccacaaagtgctccccacggggtggtatttctccacgtcacgtacgtgccagctcaggagaagaattttactacagcaccagctatatgccacgacgggaaggcccatttcccccgggaaggggtgttcgtaagcaacggtactcattggttcgttacccagcggaacttctacgagccccagattatcaccactcataataccttcgtcagcggaaattgcgacgtggtcattgggatcgtgaataatactgtgtacgacccgctccagcccgagctggatagctttaaagaagaactggacaagtacttcaagaaccacacctctccggacgtggacctgggcgatatttccggaatcaacgcatctgtggtcaatatacagaaggagatcgataggttgaacgaagtcgcaaagaacctcaacgagtcattgattgaccttcaggagttggggaaatacgaacaatacattaagtggccttggtatatctggcttggattcatcgcaggacttatcgcaattgtgatggtcaccatcatgctgtgttgcatgacgagctgttgcagctgcctgaaagggtgctgctcctgcggctcttgttgt

SEQ ID NO: 7 - DNA, 3750 bp, Unknown
  Coronavirinae
atgttcgtcttcctggtgctgctgcctttggtttcatcccaatgcgtgaacctcaccacacgtacacagctgccgccggcctatacgaactcatttaccagaggcgtctactatccggataaggtctttcggagtagcgtcctgcacagcacccaggatctgtttctgcccttcttctcaaacgtgacatggtttcacgcgatccacgtttcaggcactaacgggacgaaacgattcgccaatccggtgctccccttcaacgacggcgtgtacttcgcatcaaccgaaaagtccaatatcattcgtggttggatcttcgggaccacacttgacagtaaaactcaatctctgttgatcgtgaacaatgccaccaacgtggtaatcaaggtgtgcgagttccagttctgcaacgacccgttcctgggcgtgtactatcataagaataataagtcatggatggagagcgaatttcgggtgtactcatccgctaacaactgtaccttcgagtacgtttcacaaccgttcctcatggatcttgagggcaagcaaggcaactttaagaacctgcgggagttcgtattcaagaacatcgacggatacttcaaaatctactcaaaacatacccccataaacctcgtaagggacctgccacaaggcttctctgcgctggagcccctcgtggacctgcctatcggcatcaatattacccggttccagacactgatctcttatctcaccccgggagacagctcatctggctggacggccggggcagcagcatactatgtcgggtacctgcagccacgcacctttctgctcaagtacaacgagaacgggacaatcaccgacgcagtggattgcgccctggacccactgtccgagactaaatgcaccctcaagagctttaccgtcgagaagggtatataccagacaagcaatttccgcgtgcagcctactgagagtatcgtgcgcttcccaaacatcactaatctgtgtccattcggagaggtattcaatgcaacaaggttcgccagtgtgtacgcgtggaatcggaaacgaattagtaattgcgtggcagactacagcgttctttacaacagcgctagtttctcaaccttcaaatgctacggggtttccccgacgaagttgaacgacctgtgtttcacaaacgtgtacgctgacagtttcgtcatccggggtgacgaggttaggcagatagcccccggacagaccgggaacatcgccgactacaactacaagctccctgacgacttcacgggttgtgtaatcgcctggaattctaataacctggacagcaaagtgggcggaaactacaactatctttaccgcctgttccgtaaaagcaacctgaagccattcgaacgcgacatatctacggagatataccaagctggctctaccccatgcaacggagtgaaagggttcaactgctatttccccctgcagagctacgggtttcagccaacctacggcgtcggatatcagccctatagggtcgttgtcctgtccttcgagctgctgcacgccccggcgaccgtgtgcggcccaaagaaatcaacgaacctggtgaagaataagtgcgttaactttaattttaacggcctcactgggactggggtcttgacagaatccaataagaaattccttccctttcagcagttcggtcgcgatatcgccgatacaacagacgccgttcgggacccccaaactttggaaatactcgatatcactccctgcagtttcggcggcgtgtccgtcatcacgcctggcaccaacaccagcaatcaagtagccgtcttgtaccaaggcgtcaattgtaccgaggtgccagtcgcaatccacgccgaccagctcaccccaacgtggagagtgtactcaacgggcagcaacgtattccagaccagggcgggttgcctgatcggcgccgagcacgtgaataatagctacgaatgcgatatcccaatcggggcgggcatttgtgcctcataccaaacccaaacaaactcacccaggcgggctaggtctgttgccagccagagcataatcgcttataccatgtccctgggcgttgagaacagtgtggcctatagcaacaattccatagctattcctaccaacttcacaatctccgtaactactgagatccttcctgtctccatgactaaaacttctgtcgattgcaccatgtatatctgcggcgacagcacagagtgttcaaacctgctgctgcagtacggttccttctgcacccagctgaatagggcgttgaccggtatcgcggtcgagcaggataagaatacgcaggaagtgttcgcccaggttaagcagatttataagacgccccctatcaaggacttcggcggattcaacttctcccagattctgcctgaccccagcaagccttctaaaagatccttcatcgaagatctattgtttaataaggtaactttggccgacgccgggtttattaagcagtacggcgactgtttgggcgacatcgcagcaagggatctgatctgcgcccagaaattcaatggactgacagtgctgcccccactcttgacggacgagatgatcgcccagtataccagcgcgctcctggccggcaccattaccagtggatggacattcggcgctggcgcggctctgcagatccctttcgcaatgcagatggcgtaccgtttcaacggcataggtgtaacccaaaacgtactgtacgaaaatcagaagctgatagctaatcagttcaacagcgccatcgggaagatccaggattctctgtcaagcactgcctcagccctcgggaagttgcaggacgtagttaatcagaacgctcaggccttgaataccctcgtaaagcagttgtccagtaacttcggagctatctctagcgtgttgaacgacatattgagcaggctcgataaggtagaagccgaggtacagatcgaccgactgattaccggacgactgcagtcccttcaaacttacgtaacgcagcagctgatcagggccgccgagattagggccagcgcgaacctcgccgcaaccaagatgagtgaatgcgttctgggccagagtaagcgtgtggacttctgcggcaaaggttaccacctcatgtcttttccacaaagtgctccccacggggtggtatttctccacgtcacgtacgtgccagctcaggagaagaattttactacagcaccagctatatgccacgacgggaaggcccatttcccccgggaaggggtgttcgtaagcaacggtactcattggttcgttacccagcggaacttctacgagccccagattatcaccactgataataccttcgtcagcggaaattgcgacgtggtcattgggatcgtgaataatactgtgtacgacccgctccagcccgagctggatagctttaaagaagaactggacaagtacttcaagaaccacacctctccggacgtggacctgggcgatatttccggaatcaacgcatctgtggtcaatatacagaaggagatcgataggttgaacgaagtcgcaaagaacctcaacgagtcattgattgaccttcaggagttggggaaatacgaacaatacattaagtggccttggtatatctggcttggattcatcgcaggacttatcgcaattgtgatggtcaccatcatgctgtgttgcatgacgagctgttgcagctgcctgaaagggtgctgctcctgcggctcttgttgt

SEQ ID NO: 8 - DNA, 3756 bp, Unknown
  Coronavirinae
atgttcgtcttcctggtgctgctgcctttggtttcatcccaatgcgtgaacctcagaacacgtacacagctgccgccggcctatacgaactcatttaccagaggcgtctactatccggataaggtctttcggagtagcgtcctgcacagcacccaggatctgtttctgcccttcttctcaaacgtgacatggtttcacgcgatccacgtttcaggcactaacgggacgaaacgattcgacaatccggtgctccccttcaacgacggcgtgtacttcgcatcaaccgaaaagtccaatatcattcgtggttggatcttcgggaccacacttgacagtaaaactcaatctctgttgatcgtgaacaatgccaccaacgtggtaatcaaggtgtgcgagttccagttctgcaacgacccgttcctggacgtgtactatcataagaataataagtcatggatggagagcggagtgtactcatccgctaacaactgtaccttcgagtacgtttcacaaccgttcctcatggatcttgagggcaagcaaggcaactttaagaacctgcgggagttcgtattcaagaacatcgacggatacttcaaaatctactcaaaacatacccccataaacctcgtaagggacctgccacaaggcttctctgcgctggagcccctcgtggacctgcctatcggcatcaatattacccggttccagacactgttggccctgcaccggtcttatctcaccccgggagacagctcatctggctggacggccggggcagcagcatactatgtcgggtacctgcagccacgcacctttctgctcaagtacaacgagaacgggacaatcaccgacgcagtggattgcgccctggacccactgtccgagactaaatgcaccctcaagagctttaccgtcgagaagggtatataccagacaagcaatttccgcgtgcagcctactgagagtatcgtgcgcttcccaaacatcactaatctgtgtccattcggagaggtattcaatgcaacaaggttcgccagtgtgtacgcgtggaatcggaaacgaattagtaattgcgtggcagactacagcgttctttacaacagcgctagtttctcaaccttcaaatgctacggggtttccccgacgaagttgaacgacctgtgtttcacaaacgtgtacgctgacagtttcgtcatccggggtgacgaggttaggcagatagcccccggacagaccgggaaaatcgccgactacaactacaagctccctgacgacttcacgggttgtgtaatcgcctggaattctaataacctggacagcaaagtgggcggaaactacaactatcgttaccgcctgttccgtaaaagcaacctgaagccattcgaacgcgacatatctacggagatataccaagctggctctaaaccatgcaacggagtggaagggttcaactgctatttccccctgcagagctacgggtttcagccaaccaacggcgtcggatatcagccctatagggtcgttgtcctgtccttcgagctgctgcacgccccggcgaccgtgtgcggcccaaagaaatcaacgaacctggtgaagaataagtgcgttaactttaattttaacggcctcactgggactggggtcttgacagaatccaataagaaattccttccctttcagcagttcggtcgcgatatcgccgatacaacagacgccgttcgggacccccaaactttggaaatactcgatatcactccctgcagtttcggcggcgtgtccgtcatcacgcctggcaccaacaccagcaatcaagtagccgtcttgtaccaaggcgtcaattgtaccgaggtgccagtcgcaatccacgccgaccagctcaccccaacgtggagagtgtactcaacgggcagcaacgtattccagaccagggcgggttgcctgatcggcgccgagcacgtgaataatagctacgaatgcgatatcccaatcggggcgggcatttgtgcctcataccaaacccaaacaaactcacgcaggcgggctaggtctgttgccagccagagcataatcgcttataccatgtccctgggcgctgagaacagtgtggcctatagcaacaattccatagctattcctaccaacttcacaatctccgtaactactgagatccttcctgtctccatgactaaaacttctgtcgattgcaccatgtatatctgcggcgacagcacagagtgttcaaacctgctgctgcagtacggttccttctgcacccagctgaatagggcgttgaccggtatcgcggtcgagcaggataagaatacgcaggaagtgttcgcccaggttaagcagatttataagacgccccctatcaaggacttcggcggattcaacttctcccagattctgcctgaccccagcaagccttctaaaagatccttcatcgaagatctattgtttaataaggtaactttggccgacgccgggtttattaagcagtacggcgactgtttgggcgacatcgcagcaagggatctgatctgcgcccagaaattcaatggactgacagtgctgcccccactcttgacggacgagatgatcgcccagtataccagcgcgctcctggccggcaccattaccagtggatggacattcggcgctggcgcggctctgcagatccctttcgcaatgcagatggcgtaccgtttcaacggcataggtgtaacccaaaacgtactgtacgaaaatcagaagctgatagctaatcagttcaacagcgccatcgggaagatccaggattctctgtcaagcactgcctcagccctcgggaagttgcagaacgtagttaatcagaacgctcaggccttgaataccctcgtaaagcagttgtccagtaacttcggagctatctctagcgtgttgaacgacatattgagcaggctcgataaggtagaagccgaggtacagatcgaccgactgattaccggacgactgcagtcccttcaaacttacgtaacgcagcagctgatcagggccgccgagattagggccagcgcgaacctcgccgcaaccaagatgagtgaatgcgttctgggccagagtaagcgtgtggacttctgcggcaaaggttaccacctcatgtcttttccacaaagtgctccccacggggtggtatttctccacgtcacgtacgtgccagctcaggagaagaattttactacagcaccagctatatgccacgacgggaaggcccatttcccccgggaaggggtgttcgtaagcaacggtactcattggttcgttacccagcggaacttctacgagccccagattatcaccactgataataccttcgtcagcggaaattgcgacgtggtcattgggatcgtgaataatactgtgtacgacccgctccagcccgagctggatagctttaaagaagaactggacaagtacttcaagaaccacacctctccggacgtggacctgggcgatatttccggaatcaacgcatctgtggtcaatatacagaaggagatcgataggttgaacgaagtcgcaaagaacctcaacgagtcattgattgaccttcaggagttggggaaatacgaacaatacattaagtggccttggtatatctggcttggattcatcgcaggacttatcgcaattgtgatggtcaccatcatgctgtgttgcatgacgagctgttgcagctgcctgaaagggtgctgctcctgcggctcttgttgt

SEQ ID NO: 9 - DNA, 21 bp, Artificial sequence
  Synthetic sequence
ggatcttggttcattctcaag

SEQ ID NO: 10 - DNA, 18 bp, Artificial sequence
  Synthetic sequence
gtacctgcaaccgagaac

SEQ ID NO: 11 - DNA, 17 bp, Artificial sequence
  Synthetic sequence
ggcgttctgacggaatc

SEQ ID NO: 12 - DNA, 17 bp, Artificial sequence
  Synthetic sequence
gcaatacggtgactgcc

SEQ ID NO: 13 - DNA, 19 bp, Artificial sequence
  Synthetic sequence
cgtgtctaacggtactcac

SEQ ID NO: 14 - DNA, 18 bp, Artificial sequence
  Synthetic sequence
gttctcggttgcaggtac

SEQ ID NO: 15 - DNA, 20 bp, Artificial sequence
  Synthetic sequence
catatagacaaacgcacacc

(19) United States
(12) Patent Application Publication
(10) Pub. No.: US 2023/0296604 A1
(43) Pub. Date: Sep. 21, 2023

(54) A Method of Detecting SARS-COV2 Antibodies and Related Products

(71) Applicant: Agency for Science, Technology and Research, Singapore (SG)

(72) Inventors: Yun Shan Goh, Singapore (SG); Laurent Renia, Singapore (SG); Lisa Fong Poh Ng, Singapore (SG)

(21) Appl. No.: 18/021,445

(22) PCT Filed: Sep. 21, 2021
(86) PCT No.: PCT/SG2021/050572

§ 371 Date: Feb. 15, 2023

(30) Foreign Application Priority Data
Sep. 29, 2020 (SG) 10202009679P

(51) Int. Cl. G01N 33/569; G01N 15/14; C12Q 1/686

(52) U.S. Cl. CPC G01N 33/56983; G01N 15/14; C12Q 1/686; C12Q 2600/112; G01N 2333/165; G01N 2469/20

(57) ABSTRACT

There is provided a method of identifying/characterising coronavirus infection in a human subject. In a specific embodiment the method comprising contacting a serum sample from the subject with an isolated host cell expressing a nucleotide that encodes for a codon optimised gene for SARS-CoV-2 spike protein (S protein); and detecting a binding of an antibody or an antigen-binding fragment thereof to said host cell using a flow cytometry and a labelled anti-human secondary antibody. Also disclosed is a method of identifying a subject having immunity for coronavirus, especially SARS-CoV-2 wherein a sample from a subject is incubated with said cell and is detected using the flow cytometry and the labelled anti-human secondary antibody. Nucleic acid encoding a SARS-CoV-2 S protein, viral vectors and host cells thereof are also disclosed.

### TECHNICAL FIELD

The present disclosure relates broadly to methods of detecting antibodies in a subject and related products thereof.

### BACKGROUND

The coronavirus disease 2019 (COVID-19) is a major health issue affecting 216 countries, with 22 million confirmed human infection cases and 800,000 fatalities (WHOref) thus far. By April 2020, a mere four months after the first case of COVID-19 was reported, COVID-19 has developed into a pandemic that has led to either a partial or total confinement of more than half of the world's population. This unprecedented crisis has resulted in overwhelmed healthcare systems and major socio-economic disruptions.

One of the key public health strategies to control the spread of COVID-19 is to have early detection of infected individuals to allow early blocking of the transmission of Severe Acute Respiratory Syndrome Coronavirus (SARS-CoV-2). Although quantitative reverse transcriptase PCR (qRT-PCR) remain the gold standard for COVID-19 diagnosis, it can be time-consuming and the incidences of false negative diagnosis can occur due to insufficient viral genetic material at the point of detection, either inefficient sample preparation or low viral load in the patients. SARS-CoV-2 viral RNA becomes almost undetectable in most patients by 14 days post-illness onset (pio).

Serological assays that detect specific antibodies against SARS-CoV-2 have increasingly been utilised to complement PCR-based assays, especially in symptomatic infections with low viral load. As of 17 Aug. 2020, the U.S. Food and Drug Administration (US-FDA) approved the use of 37 serological assays (US-FDA, 2020), mainly targeting two immunogenic proteins: (1) spike (S) protein, which is the most exposed viral protein, and (2) nucleocapsid (N) protein, which is abundantly expressed during infection. The S protein is responsible for the binding and entry of the virus into the host cell via the cellular receptor, angiotensin-converting enzyme 2 (ACE2). Many S protein-based serological assays detect specific antibodies to either the S1 subunit, S2 subunit or the receptor binding domain (RBD), which do not allow the capture of full repertoire of antibodies such as antibodies binding to various domains and conformational epitopes of the S protein.

Serological diagnosis is especially important for asymptomatic patients and patients with mild to moderate illness who may have a late onset of illness. Serological diagnosis is also critical to identify individuals who are immune and potentially protected. Thus, there is a need to provide an alternative method of identifying/characterising coronavirus infection in a subject. In particular, there is a need to provide an alternative method that detects SARS-CoV2 antibodies in subjects.

### SUMMARY

In one aspect, there is provided a method of identifying/characterizing coronavirus infection, optionally SARS-CoV-2 infection in a subject, the method comprising: contacting a sample from the subject with an isolated host cell comprising a nucleic acid encoding for the spike protein (S protein) of the coronavirus; and detecting a binding of an antibody or fragment thereof to the host cell.

In another aspect, there is provided a method of identifying a subject having immunity for coronavirus infection, optionally SARS-CoV-2 infection, the method comprising: contacting/incubating a sample from the subject with an isolated host cell comprising a nucleic acid encoding for the spike protein (S protein) of the coronavirus; and detecting a binding of an antibody or fragment thereof to the host cell.

In some embodiments, the detecting step comprises using a fluorescence detection instrument, optionally wherein the instrument is a flow cytometer to detect a binding of antibody or fragment thereof to the host cell.

In some embodiments, the method further comprising contacting/incubating the sample with one or more detection or secondary antibody.

In some embodiments, the method is a diagnostic method, optionally a serological diagnostic method.

In some embodiments, the method further comprising performing a further assay such as a polymerase chain reaction (PCR)-based assay to detect/confirm coronavirus infection, optionally SARS-CoV-2 infection. In some embodiments, the method is an in-vitro or an ex-vivo method.

In yet another aspect, there is provided a nucleic acid, optionally an isolated and/or recombinant nucleic acid, encoding for the spike protein (S protein) of severe acute respiratory syndrome (SARS)-like coronavirus (SARS-CoV-2).

In yet another aspect, there is provided a viral vector, optionally a recombinant viral vector, comprising the nucleic acid as described herein.

In yet another aspect, there is provided a host cell, optionally an isolated host cell, comprising the nucleic acid as described herein, optionally wherein the host cell is transduced with the viral vector as described herein.

In some embodiments, the host cell expresses the S protein.

In some embodiments, the S protein comprises a full-length S protein.

In some embodiments, the S protein/the full-length S protein comprises SEQ ID NO: 1, SEQ ID NO: 3, SEQ ID NO: 4, SEQ ID NO: 5, or a sequence sharing at least about 75% sequence identity with SEQ ID NO: 1, SEQ ID NO: 3, SEQ ID NO: 4, or SEQ ID NO: 5.

In some embodiments, the nucleic acid comprises SEQ ID NO: 2, SEQ ID NO: 6, SEQ ID NO: 7, SEQ ID NO: 8, or a sequence sharing at least about 75% sequence identity with SEQ ID NO: 2, SEQ ID NO: 6, SEQ ID NO: 7, or SEQ ID NO: 8.

In some embodiments, the S protein/the full-length S protein comprises SEQ ID NO: 1 or a sequence sharing at least about 75% sequence identity with SEQ ID NO. 1.

In some embodiments, the nucleic acid comprises SEQ ID NO: 2, or a sequence sharing at least about 75% sequence identity with SEQ ID NO: 2.

In some embodiments, the host cells bound to an antigen binding protein or a fragment thereof, optionally an antibody, further optionally an antibody against SARS-CoV-2.

### Definitions

The term “antigen binding protein” as used herein broadly refers to any peptide-based molecule that recognizes and binds to a target such as a S protein-expressing cell. Examples of antigen binding proteins include antibodies, including an antibody of any of the five major classes of immunoglobulins: IgA, IgD, IgE, IgG, and IgM, or subclasses (isotypes) thereof (e.g. IgG1, IgG2, IgG3, IgG4, IgA1 and IgA2), and antigen-binding fragments thereof. In some examples, the antigen binding protein comprises anti-spike protein IgA, IgG (including IgG1, IgG2, IgG3, IgG4) and IgM.

The term “identifying” or “characterizing” as used herein in relation to an infection is to be interpreted broadly to encompass determining a presence, an absence, an amount, or a level of disease burden/severity of the infection. Identifying or characterizing an infection may also include identifying/estimating/predicting a previous infection, a stage of the infection (early stage, late stage, recovery stage, a convalescent stage etc.), an outcome/prognosis of the infection (e.g. chances of recovery, death etc.), measuring an immune response against the infection and/or measuring a ratio/proportion of a particular class/subclass of immunoglobulin as compared to the total immunoglobulin or total immunoglobulin in a class. For example, a proportion of IgG1 response as compared to the total IgG response may be measured.

For example, an antibody response may indicate the presence of an infection. For example, a strong antibody response (e.g. exceeding a threshold) may indicate that the infection is in a late stage and/or the disease is more severe. For example, a strong antibody response (e.g. a strong IgM response and/or IgG response including IgG1, IgG2, IgG3 and IgG4) is correlated, optionally positively correlated, with one or more of disease severity, pneumonia, more severe clinical outcomes such as oxygen supply requirement and ICU admission. In various examples, the correlation is observed at about 10 days pio and/or about 23 days pio. For example, a positive IgG1 and/or IgG3 response, which are important in neutralizing virus, may be indicative of prognosis in a subject.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### Description of Embodiments

Exemplary, non-limiting embodiments of a method of identifying/characterising coronavirus infection in a subject are disclosed hereinafter.

In another aspect, there is provided a nucleic acid, optionally an isolated and/or recombinant nucleic acid, encoding for the spike protein (S protein) of severe acute respiratory syndrome (SARS)-like coronavirus (SARS-CoV-2).

In various embodiments, the nucleic acid comprises deoxyribonucleic acid (DNA) and/or ribonucleic acid (RNA). The nucleic acid may be single stranded or double stranded.

In various embodiments, the nucleic acid further comprises a marker gene. A marker gene may be a gene that confers ability to select a cell so that only the cell expressing the gene can be selected. Examples of marker genes include, but are not limited to, antibiotic/drug resistance genes, fluorescence-activated cell sorting (FACS)-detectable genes, enzyme genes and genes encoding for a fluorescent protein and the like. Examples of a fluorescent protein include, but are not limited to, green fluorescent protein (GFP), firefly luciferase, bacterial luciferase, phycobiliproteins and the like. In one embodiment, the nucleic acid comprises GFP gene.

In various embodiments, the nucleic acid further comprises an internal ribosome entry site (IRES) sequence.

In various embodiments, the nucleic acid comprises a plasmid.

In various embodiments, the nucleic acid is devoid of or does not comprise or lacks one of more of the other genes/sequences (other than the gene/sequence encoding for S protein) of SARS-CoV-2. In various embodiments, the nucleic acid is devoid of or does not comprise or lacks the genes/sequences encoding for one or more of the other SARS-CoV-2 proteins (other than the S protein). In some embodiments, the nucleic acid is devoid of or does not comprise or lacks one of more of the other genes that encode the viral proteins necessary for replication, transcription and infectious virus assembly. In some embodiments, the nucleic acid is devoid of or does not comprise or lacks the genes/sequences encoding for one or more of the other structural proteins including small envelope (E) glycoprotein, membrane (M) glycoprotein and nucleocapsid (N) protein, the other accessory proteins and the other non-structural proteins including nsp1 through nsp16.

In various embodiments, the nucleic acid comprises a SARS-CoV-2 gene/sequence or a SARS-CoV-2-derived gene/sequence or a SARS-CoV-2-associated gene/sequence consisting of a gene/sequence encoding for the S protein. In various embodiments, the nucleic acid encodes for a SARS-CoV-2 protein consisting of the S protein.

In yet another aspect, there is provided a viral vector, optionally a recombinant viral vector, comprising the nucleic acid as described herein.

Examples of viral vectors include, but are not limited to retroviral vector, lentiviral vector, adenoviral vector, adeno-associated viral vector (AAV), hybrid vectors (e.g. adeno-retro hybrid viral vector and adeno-lenti hybrid viral vector) and the like. In various embodiments, the viral vector comprises a retroviral vector. In various embodiments, the retroviral vector comprises a lentiviral vector.

The viral vector may be used to transduce a wide range of cells that can support the expression of the S protein. Examples of host cells include, but are not limited to primary cells, immortalized cells and embryonic stem cells.

In various embodiments, there is provided a virus, optionally a recombinant virus, comprising the nucleic acid as described herein. Examples of viruses include, but are not limited to, retrovirus, lentivirus, adenovirus, adeno-associated virus (AAV), hybrids (e.g. adeno-retro hybrid and adeno-lenti hybrid) and the like.

The virus or the viral vector may be a recombinant virus or a recombinant viral vector. In various embodiments, the virus or the viral vector does not comprise coronavirus such as SARS-CoV-2.

In various embodiments, the virus or the viral vector is incapable of replication or replication-incompetent.

In yet another aspect, there is provided a host cell, optionally an isolated host cell, comprising the nucleic acid as described herein, optionally wherein the host cell is transduced with the viral vector as described herein.

In various embodiments, the host cell comprises a mammalian cell. In various embodiments, the mammalian cell comprises a human cell. In various embodiments, the host cell comprises a human embryonic kidney (HEK) cell/cell line or derivatives thereof. In various embodiments, the host cell comprises HEK 293, optionally HEK 293T cells.

In various embodiments, the nucleic acid is integrated into the host cell genome (e.g. stable transfection). In various embodiments, the host cell is derived from a cell line such as a stable cell line.

In various embodiments, the host cell expresses the S protein.

In various embodiments, the S protein comprises a receptor-binding subunit (S1) and/or a membrane fusion subunit (S2). In various embodiments, the S protein comprises a N-terminal domain (NTD), a receptor-binding domain (RBD), a potential fusion peptide (pFP)/fusion peptide (FP), a heptad repeat-N(HR-N), a heptad repeat-C (HR-C), a transmembrane domain (TM) and/or an intracellular tail (IC). In various embodiments, the S protein comprises a trimer.

In various embodiments, the S protein is accessible or exposed on the cell surface.

In various embodiments, the host cell further expresses a fluorescent protein. In various embodiments, the host cell is GFP-positive.

In various embodiments, the host cell is capable of capturing a full repertoire of specific antibodies against the S protein. For example, embodiments of the host cell is capable of capturing not only antibodies against the receptor-binding domain (RBD) of the S protein, but also antibodies against other domains of the S protein and/or conformational epitopes of the S protein.

In various embodiments, the viral vector or the host cell of present disclosure, wherein the S protein comprises a full-length S protein.

In various embodiments, wherein the S protein/the full-length S protein comprises SEQ ID NO: 1, SEQ ID NO: 3, SEQ ID NO: 4, SEQ ID NO: 5 or a sequence sharing at least about 75% sequence identity with SEQ ID NO: 1, SEQ ID NO: 3, SEQ ID NO: 4, or SEQ ID NO: 5.

SEQ ID NO: 1
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQTYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
SEQ ID NO: 3
MFVFLVLLPLVSSQCVNLTTRTQLPPAYTNSFTRGVYYPDKVFRSSVLHSTQDLFLPFFS
NVTWFHAISGTNGTKRFDNPVLPFNDGVYFASTEKSNIIRGWIFGTTLDSKTQSLLIVNN
ATNVVIKVCEFQFCNDPFLGVHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTLK
SFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTYGVGYQPYRVVVLSFELLHAPATVCGPKKSTNLVKNKCVNFNFN
GLTGTGVLTESNKKFLPFQQFGRDIDDTTDAVRDPQTLEILDITPCSFGGVSVITPGTNT
SNQVAVLYQGVNCTEVPVAIHADQLTPTWRVYSTGSNVFQTRAGCLIGAEHVNNSYECDI
PIGAGICASYQTQTNSHRRARSVASQSIIAYTMSLGAENSVAYSNNSIAIPINFTISVTT
EILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGFIKQYGDCLGDI
AARDLICAQKFNGLTVLPPLLTDEMIAQYTSALLAGTITSGWTFGAGAALQIPFAMQMAY
RFNGIGVTQNVLYENQKLIANQFNSAIGKIQDSLSSTASALGKLQDVVNQNAQALNTLVK
QLSSNFGAISSVLNDILARLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHD
GKAHFPREGVFVSNGTHWFVTQRNFYEPQIITTHNTFVSGNCDVVIGIVNNTVYDPLQPE
LDSFKEELDKYFKNHTSPDVDLGDISGINASVVNIQKEIDRLNEVAKNLNESLIDLQELG
KYEQYIKWPWYIWLGFIAGLIAIVMVTIMLCCMTSCCSCLKGCCSCGSCC
SEQ ID NO: 4
MFVFLVLLPLVSSQCVNLTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LISYLTPGDSSSGWTAGAAAYYVGYLQPRTFLLKYNENGTITDAVDCALDPLSETKCTLK
SFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGNIADYNYK
LPDDFTGCVIAWNSNNLDSKVGGNYNYLYRLFRKSNLKPFERDISTEIYQAGSTPCNGVK
GFNCYFPLQSYGFQPTYGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNT
SNQVAVLYQGVNCTEVPVAIHADQLTPTWRVYSTGSNVFQTRAGCLIGAEHVNNSYECDI
PIGAGICASYQTQTNSPRRARSVASQSIIAYTMSLGVENSVAYSNNSIAIPTNFTISVTT
EILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGFIKQYGDCLGDI
AARDLICAQKFNGLTVLPPLLTDEMIAQYTSALLAGTITSGWTFGAGAALQIPFAMQMAY
RFNGIGVTQNVLYENQKLIANQFNSAIGKIQDSLSSTASALGKLQDVVNQNAQALNTLVK
QLSSNFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHD
GKAHFPREGVFVSNGTHWFVTQRNFYEPQIITTDNTFVSGNCDVVIGIVNNTVYDPLQPE
LDSFKEELDKYFKNHTSPDVDLGDISGINASVVNIQKEIDRLNEVAKNLNESLIDLQELG
KYEQYIKWPWYIWLGFIAGLIAIVMVTIMLCCMTSCCSCLKGCCSCGSCC
SEQ ID NO: 5
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIADYN
YKLPDDFTGCVIAWNSNNLDSKVGGNYNYRYRLFRKSNLKPFERDISTEIYQAGSKPCNG
VEGFNCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEHVNNSYEC
DIPIGAGICASYQTQTNSRRRARSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVF
AQVKQTYKTPPIKDFGGFNFSQILPDPSKPSKRSFIEDLLFNKVTLADAGFIKQYGDCLG
DIAARDLICAQKFNGLTVLPPLLTDEMIAQYTSALLAGTITSGWTFGAGAALQIPFAMQM
AYRENGIGVTQNVLYENQKLIANQFNSAIGKIQDSLSSTASALGKLQNVVNQNAQALNTL
VKQLSSNFGAISSVLNDILSRLDKVEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQ
PELDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCC

In various embodiments, the S protein/the full-length S protein comprises a sequence sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity with SEQ ID NO: 1, SEQ ID NO: 3, SEQ ID NO: 4, and/or SEQ ID NO: 5. In some embodiments, the S protein/the full-length S protein comprises a sequence sharing at least about 99.1%, at least about 99.2%, at least about 99.3%, at least about 99.4%, at least about 99.5%, at least about 99.6%, at least about 99.7%, at least about 99.8% or at least about 99.9% sequence identity with SEQ ID NO: 1, SEQ ID NO: 3, SEQ ID NO: 4, and/or SEQ ID NO: 5.

In various embodiments, the S protein/the full-length S protein comprises no less than about 1300 amino acid residues, no less than about 1250 amino acid residues, no less than about 1200 amino acid residues, no less than about 1150 amino acid residues, no less than about 1100 amino acid residues, no less than about 1050 amino acid residues or no less than about 1000 amino acid residues. In various embodiments, the S protein/the full-length S protein comprises no less than about 1280 amino acid residues, no less than about 1279 amino acid residues, no less than about 1278 amino acid residues, no less than about 1277 amino acid residues, no less than about 1276 amino acid residues, no less than about 1275 amino acid residues, no less than about 1274 amino acid residues, no less than about 1273 amino acid residues, no less than about 1272 amino acid residues, no less than about 1271 amino acid residues, no less than about 1270 amino acid residues, no less than about 1269 amino acid residues, no less than about 1268 amino acid residues, no less than about 1267 amino acid residues, no less than about 1266 amino acid residues, no less than about 1265 amino acid residues, no less than about 1264 amino acid residues, no less than about 1263 amino acid residues, no less than about 1262 amino acid residues, no less than about 1261 amino acid residues, no less than about 1260 amino acid residues, no less than about 1259 amino acid residues, no less than about 1258 amino acid residues, no less than about 1257 amino acid residues, no less than about 1256 amino acid residues, no less than about 1255 amino acid residues, no less than about 1254 amino acid residues, no less than about 1253 amino acid residues, no less than about 1252 amino acid residues, no less than about 1251 amino acid residues or no less than about 1250 amino acid residues. In various embodiments, the S protein/the full-length S protein comprises from about 1000 to about 1500 amino acid residues, from about 1100 to about 1400 amino acid residues, from about 1200 to about 1300 amino acid residues, from about 1250 to about 1300 amino acid residues, from about 1260 to about 1280 amino acid residues, from about 1270 to about 1280 amino acid residues or from about 1270 to about 1275 amino acid residues. In one embodiment, the S protein/the full-length S protein comprises about 1273 amino acid residues.

In various embodiments, the S protein/the full-length S protein includes a protein comprising one or more mutation(s) e.g. deletion(s), insertion(s), and/or substitution(s) with other amino acid(s), e.g. with conservative substitutions as compared to SEQ ID NO: 1, SEQ ID NO: 3, SEQ ID NO: 4, and/or SEQ ID NO: 5. Conservative substitution tables are well known in the art, useful substitutions can be found by the skilled person by using such tables or by using routine experimentation. Examples for conservative substitutions are those wherein the amino acids which originate from the same class or which have similar biochemical properties are exchanged for each other. For example, a basic amino acid may be substituted with another basic amino acid, an acidic amino acid may be substituted with another acidic amino acid, an aromatic amino acid may be substituted with another aromatic amino acid, a non-polar, aliphatic amino acid may be substituted with another non-polar, aliphatic amino acid and a polar, uncharged amino acid may be substituted with another polar, uncharged amino acid etc.

In various embodiments, the mutation(s) have minimal or no impact on the function/activity of the S protein/the full-length S protein. In various embodiments, the mutation(s) have minimal or no impact on the structure or folding of the protein/the full-length S protein. In various embodiments, the mutation(s) have minimal or no impact on the antigenic properties of the S protein/the full-length S protein. In various embodiments, where the mutation(s) results in a variant of an epitope/domain, the variant is a functional variant of the epitope/domain. For example, the functional variant epitope/domain is capable of being specifically recognised and/or bound by the same antibody/antigen binding protein/antigen-binding fragment as the unmodified/unmutated epitope/domain to at least substantially the same level.

Methods which can be used to determine the antigenic properties of a protein or recognition (or amount of recognition) by a variant epitope/domain are known in the art. For example, an ELISA assay can be used to compare a level of specific binding of an antibody to a S protein/full-length S protein to a level of specific binding of the antibody to a corresponding S protein/full-length S protein comprising mutation(s) to determine if the mutation(s) affect the specific binding of the mutated protein to the antibody.

In various embodiments, the S protein/the full-length S protein comprises from about 1 to about 200 mutation(s), from about 1 to about 150 mutation(s), from about 1 to about 100 mutation(s), from about 1 to about 50 mutation(s) or from about 1 to about 20 mutation(s).

In various embodiments, the S protein/the full-length S protein includes the S protein from a variant of SARS-CoV2 virus. For example, the S protein/the full-length S protein is the S protein from an alpha variant (SEQ ID NO: 3), a beta variant (SEQ ID NO: 4), a delta variant (SEQ ID NO: 5), and the like.

In various embodiments, the nucleic acid comprises SEQ ID NO: 2, SEQ ID NO: 6, SEQ ID NO: 7, SEQ ID NO: 8 or a sequence sharing at least about 75% sequence identity with SEQ ID NO: 2, SEQ ID NO: 6, SEQ ID NO: 7, SEQ ID NO: 8.

SEQ ID NO: 2
ATGTTTGTATTCTTGGTACTTCTCCCATTGGTATCTTCTCAATGCGTTAACCTTACCACACGC
ACCCAACTGCCCCCGGCCTACACTAATAGCTTTACGCGGGGTGTCTACTATCCCGACAAAGTC
TTTCGATCCAGTGTGCTCCACTCCACCCAGGATCTTTTCCTTCCCTTTTTTTCTAATGTTACG
TGGTTCCACGCAATCCATGTATCCGGTACGAATGGGACAAAACGCTTTGACAATCCAGTGCTG
CCATTTAATGATGGAGTGTACTTTGCATCTACCGAGAAGAGTAACATCATCAGAGGATGGATC
TTCGGAACGACCTTGGACTCCAAAACGCAATCCTTGCTTATCGTTAACAATGCAACGAATGTT
GTCATCAAAGTTTGCGAATTCCAATTCTGTAACGATCCCTTCCTCGGTGTTTATTATCATAAA
AATAATAAATCTTGGATGGAAAGTGAGTTCCGCGTATACAGTTCCGCCAATAATTGTACCTTC
GAATACGTAAGTCAACCGTTCTTGATGGATCTGGAAGGTAAACAGGGTAACTTTAAGAACCTT
CGGGAGTTTGTTTTTAAGAACATAGACGGCTACTTTAAGATCTATAGTAAACATACGCCAATT
AACTTGGTTAGAGATCTCCCGCAGGGGTTTTCAGCATTGGAGCCGCTCGTCGACCTCCCCATA
GGTATAAATATAACTCGGTTTCAAACACTGCTGGCGCTCCACCGCAGCTACCTGACGCCTGGG
GATTCTTCTTCCGGTTGGACTGCAGGCGCTGCTGCATATTATGTAGGGTACCTGCAACCGAGA
ACCTTTCTCCTTAAGTACAACGAGAATGGCACTATTACGGACGCTGTCGATTGTGCACTCGAC
CCCTTGAGTGAGACGAAGTGTACACTGAAAAGCTTTACTGTTGAAAAGGGAATATATCAGACA
TCCAACTTTAGAGTTCAGCCAACAGAATCCATCGTTCGATTTCCCAATATTACAAATCTCTGT
CCGTTCGGAGAGGTCTTTAATGCTACCCGATTCGCGTCAGTATACGCCTGGAACAGAAAGAGA
ATTTCTAACTGTGTTGCAGATTATAGTGTCCTGTATAATTCTGCGTCTTTTAGCACTTTTAAG
TGCTACGGCGTTAGCCCCACTAAGTTGAACGACCTTTGTTTCACTAACGTGTATGCCGACTCA
TTCGTCATAAGAGGCGACGAAGTTAGACAAATTGCACCGGGCCAGACGGGAAAGATTGCGGAC
TACAACTATAAATTGCCTGACGACTTTACAGGATGTGTCATCGCCTGGAATAGTAATAACCTT
GACTCCAAAGTCGGTGGCAATTACAATTACTTGTACCGGCTGTTCAGGAAGTCTAATCTCAAA
CCTTTTGAGCGAGATATCAGCACGGAAATTTATCAAGCTGGTAGCACTCCATGTAACGGGGTT
GAGGGTTTTAATTGTTATTTTCCATTGCAATCATATGGATTCCAACCGACTAACGGTGTTGGG
TATCAACCATACAGAGTGGTGGTTTTGTCATTTGAACTTCTGCATGCCCCTGCAACAGTGTGC
GGACCGAAGAAGAGTACGAACCTTGTAAAGAACAAGTGCGTCAACTTCAACTTTAATGGTCTG
ACGGGTACCGGCGTTCTGACGGAATCCAATAAAAAGTTCTTGCCCTTTCAGCAGTTCGGGCGA
GATATCGCCGACACTACTGATGCGGTGCGAGATCCTCAGACACTTGAGATCCTCGATATTACC
CCATGTAGTTTTGGTGGTGTGTCTGTGATTACACCCGGCACCAATACGTCAAATCAGGTCGCA
GTCTTGTACCAAGACGTGAACTGCACCGAAGTTCCTGTAGCCATTCACGCTGATCAATTGACA
CCGACATGGAGGGTGTACTCCACCGGATCTAACGTGTTCCAGACCCGCGCGGGGTGTCTTATC
GGCGCAGAACATGTGAACAACTCTTACGAATGTGATATTCCTATCGGTGCAGGCATCTGTGCC
TCATACCAGACACAAACGAACTCACCAAGGAGGGCAAGGTCAGTAGCCTCACAAAGCATAATA
GCCTATACGATGAGTCTTGGTGCGGAGAACTCTGTGGCGTACTCTAATAACTCTATCGCCATA
CCGACTAACTTCACCATTTCTGTTACGACCGAGATCCTCCCAGTTTCCATGACTAAGACAAGT
GTGGATTGTACAATGTACATCTGCGGCGACAGTACTGAGTGCAGTAACCTGCTTCTGCAGTAC
GGGTCCTTCTGCACACAACTTAACCGGGCGCTGACTGGTATAGCGGTTGAACAAGACAAGAAC
ACTCAAGAGGTCTTCGCACAAGTAAAACAAATATACAAAACACCACCTATTAAAGATTTCGGC
GGGTTTAATTTTAGCCAAATCCTTCCAGACCCCAGCAAACCCTCTAAGCGCAGCTTCATTGAG
GATCTGCTGTTTAACAAGGTCACCCTGGCAGACGCGGGCTTTATCAAGCAATACGGTGACTGC
CTGGGGGATATCGCGGCTCGAGACCTTATATGTGCGCAAAAATTTAATGGACTTACCGTACTT
CCTCCATTGCTGACTGACGAGATGATAGCACAGTATACATCTGCACTGCTCGCCGGTACAATT
ACATCAGGGTGGACATTTGGGGCGGGAGCTGCGCTCCAGATACCGTTCGCGATGCAGATGGCG
TATAGGTTTAATGGAATTGGTGTCACGCAAAACGTTCTCTATGAAAACCAGAAGCTGATAGCA
AATCAGTTCAATTCCGCGATTGGTAAGATACAAGATTCATTGTCTAGTACGGCCTCTGCACTC
GGAAAACTCCAAGATGTAGTGAACCAAAACGCCCAAGCCCTGAATACACTCGTAAAACAGCTC
TCTAGTAATTTTGGGGCCATTTCCTCCGTATTGAACGACATCTTGAGTCGCTTGGATAAGGTA
GAAGCAGAAGTACAAATTGACCGGTTGATCACGGGCAGACTTCAATCACTTCAGACTTATGTT
ACTCAGCAGCTTATACGAGCTGCAGAAATTCGCGCCTCTGCGAACCTGGCCGCCACTAAAATG
TCAGAATGTGTACTGGGACAGAGCAAACGGGTGGATTTCTGCGGAAAGGGCTATCATCTGATG
AGTTTTCCCCAGTCTGCGCCTCATGGTGTAGTATTTCTTCATGTCACATATGTACCAGCCCAA
GAAAAAAATTTCACAACGGCGCCCGCGATTTGCCATGACGGTAAGGCGCATTTTCCTCGCGAG
GGCGTTTTCGTGTCTAACGGTACTCACTGGTTCGTAACACAGCGAAACTTTTACGAGCCTCAG
ATAATCACGACGGATAACACATTTGTCTCCGGCAACTGCGATGTGGTCATCGGTATAGTGAAC
AATACGGTATATGATCCGCTGCAGCCAGAGCTCGACAGTTTCAAGGAGGAGCTTGACAAATAC
TTTAAGAACCATACCTCCCCAGACGTAGACCTCGGAGACATATCTGGTATCAATGCCTCCGTG
GTTAACATACAAAAGGAGATAGATAGACTGAATGAGGTGGCGAAGAATCTGAATGAGTCTCTC
ATAGATCTGCAGGAACTCGGTAAATATGAACAATACATCAAGTGGCCTTGGTACATCTGGCTG
GGGTTCATAGCGGGCCTGATCGCGATCGTGATGGTAACTATAATGTTGTGTTGCATGACCTCC
TGCTGCTCATGCCTTAAAGGTTGTTGTTCTTGCGGGAGCTGCTGCAAGTTCGATGAGGATGAT
TCAGAACCCGTCTTGAAGGGCGTAAAACTTCACTATACGTAA
SEQ ID NO: 6
ATGTTCGTCTTCCTGGTGCTGCTGCCTTTGGTTTCATCCCAATGCGTGAACCTCACCACACGT
ACACAGCTGCCGCCGGCCTATACGAACTCATTTACCAGAGGCGTCTACTATCCGGATAAGGTC
TTTCGGAGTAGCGTCCTGCACAGCACCCAGGATCTGTTTCTGCCCTTCTTCTCAAACGTGACA
TGGTTTCACGCGATCTCAGGCACTAACGGGACGAAACGATTCGACAATCCGGTGCTCCCCTTC
AACGACGGCGTGTACTTCGCATCAACCGAAAAGTCCAATATCATTCGTGGTTGGATCTTCGGG
ACCACACTTGACAGTAAAACTCAATCTCTGTTGATCGTGAACAATGCCACCAACGTGGTAATC
AAGGTGTGCGAGTTCCAGTTCTGCAACGACCCGTTCCTGGGCGTGCATAAGAATAATAAGTCA
TGGATGGAGAGCGAATTTCGGGTGTACTCATCCGCTAACAACTGTACCTTCGAGTACGTTTCA
CAACCGTTCCTCATGGATCTTGAGGGCAAGCAAGGCAACTTTAAGAACCTGCGGGAGTTCGTA
TTCAAGAACATCGACGGATACTTCAAAATCTACTCAAAACATACCCCCATAAACCTCGTAAGG
GACCTGCCACAAGGCTTCTCTGCGCTGGAGCCCCTCGTGGACCTGCCTATCGGCATCAATATT
ACCCGGTTCCAGACACTGTTGGCCCTGCACCGGTCTTATCTCACCCCGGGAGACAGCTCATCT
GGCTGGACGGCCGGGGCAGCAGCATACTATGTCGGGTACCTGCAGCCACGCACCTTTCTGCTC
AAGTACAACGAGAACGGGACAATCACCGACGCAGTGGATTGCGCCCTGGACCCACTGTCCGAG
ACTAAATGCACCCTCAAGAGCTTTACCGTCGAGAAGGGTATATACCAGACAAGCAATTTCCGC
GTGCAGCCTACTGAGAGTATCGTGCGCTTCCCAAACATCACTAATCTGTGTCCATTCGGAGAG
GTATTCAATGCAACAAGGTTCGCCAGTGTGTACGCGTGGAATCGGAAACGAATTAGTAATTGC
GTGGCAGACTACAGCGTTCTTTACAACAGCGCTAGTTTCTCAACCTTCAAATGCTACGGGGTT
TCCCCGACGAAGTTGAACGACCTGTGTTTCACAAACGTGTACGCTGACAGTTTCGTCATCCGG
GGTGACGAGGTTAGGCAGATAGCCCCCGGACAGACCGGGAAAATCGCCGACTACAACTACAAG
CTCCCTGACGACTTCACGGGTTGTGTAATCGCCTGGAATTCTAATAACCTGGACAGCAAAGTG
GGCGGAAACTACAACTATCTTTACCGCCTGTTCCGTAAAAGCAACCTGAAGCCATTCGAACGC
GACATATCTACGGAGATATACCAAGCTGGCTCTACCCCATGCAACGGAGTGGAAGGGTTCAAC
TGCTATTTCCCCCTGCAGAGCTACGGGTTTCAGCCAACCtACGGCGTCGGATATCAGCCCTAT
AGGGTCGTTGTCCTGTCCTTCGAGCTGCTGCACGCCCCGGCGACCGTGTGCGGCCCAAAGAAA
TCAACGAACCTGGTGAAGAATAAGTGCGTTAACTTTAATTTTAACGGCCTCACTGGGACTGGG
GTCTTGACAGAATCCAATAAGAAATTCCTTCCCTTTCAGCAGTTCGGTCGCGATATCGaCGAT
ACAACAGACGCCGTTCGGGACCCCCAAACTTTGGAAATACTCGATATCACTCCCTGCAGTTTC
GGCGGCGTGTCCGTCATCACGCCTGGCACCAACACCAGCAATCAAGTAGCCGTCTTGTACCAA
GgCGTCAATTGTACCGAGGTGCCAGTCGCAATCCACGCCGACCAGCTCACCCCAACGTGGAGA
GTGTACTCAACGGGCAGCAACGTATTCCAGACCAGGGCGGGTTGCCTGATCGGCGCCGAGCAC
GTGAATAATAGCTACGAATGCGATATCCCAATCGGGGCGGGCATTTGTGCCTCATACCAAACC
CAAACAAACTCACaCAGGCGGGCTAGGTCTGTTGCCAGCCAGAGCATAATCGCTTATACCATG
TCCCTGGGCGCTGAGAACAGTGTGGCCTATAGCAACAATTCCATAGCTATTCCTAtCAACTTC
ACAATCTCCGTAACTACTGAGATCCTTCCTGTCTCCATGACTAAAACTTCTGTCGATTGCACC
ATGTATATCTGCGGCGACAGCACAGAGTGTTCAAACCTGCTGCTGCAGTACGGTTCCTTCTGC
ACCCAGCTGAATAGGGCGTTGACCGGTATCGCGGTCGAGCAGGATAAGAATACGCAGGAAGTG
TTCGCCCAGGTTAAGCAGATTTATAAGACGCCCCCTATCAAGGACTTCGGCGGATTCAACTTC
TCCCAGATTCTGCCTGACCCCAGCAAGCCTTCTAAAAGATCCTTCATCGAAGATCTATTGTTT
AATAAGGTAACTTTGGCCGACGCCGGGTTTATTAAGCAGTACGGCGACTGTTTGGGCGACATC
GCAGCAAGGGATCTGATCTGCGCCCAGAAATTCAATGGACTGACAGTGCTGCCCCCACTCTTG
ACGGACGAGATGATCGCCCAGTATACCAGCGCGCTCCTGGCCGGCACCATTACCAGTGGATGG
ACATTCGGCGCTGGCGCGGCTCTGCAGATCCCTTTCGCAATGCAGATGGCGTACCGTTTCAAC
GGCATAGGTGTAACCCAAAACGTACTGTACGAAAATCAGAAGCTGATAGCTAATCAGTTCAAC
AGCGCCATCGGGAAGATCCAGGATTCTCTGTCAAGCACTGCCTCAGCCCTCGGGAAGTTGCAG
GACGTAGTTAATCAGAACGCTCAGGCCTTGAATACCCTCGTAAAGCAGTTGTCCAGTAACTTC
GGAGCTATCTCTAGCGTGTTGAACGACATATTGgcCAGGCTCGATAAGGTAGAAGCCGAGGTA
CAGATCGACCGACTGATTACCGGACGACTGCAGTCCCTTCAAACTTACGTAACGCAGCAGCTG
ATCAGGGCCGCCGAGATTAGGGCCAGCGCGAACCTCGCCGCAACCAAGATGAGTGAATGCGTT
CTGGGCCAGAGTAAGCGTGTGGACTTCTGCGGCAAAGGTTACCACCTCATGTCTTTTCCACAA
AGTGCTCCCCACGGGGTGGTATTTCTCCACGTCACGTACGTGCCAGCTCAGGAGAAGAATTTT
ACTACAGCACCAGCTATATGCCACGACGGGAAGGCCCATTTCCCCCGGGAAGGGGTGTTCGTA
AGCAACGGTACTCATTGGTTCGTTACCCAGCGGAACTTCTACGAGCCCCAGATTATCACCACT
cATAATACCTTCGTCAGCGGAAATTGCGACGTGGTCATTGGGATCGTGAATAATACTGTGTAC
GACCCGCTCCAGCCCGAGCTGGATAGCTTTAAAGAAGAACTGGACAAGTACTTCAAGAACCAC
ACCTCTCCGGACGTGGACCTGGGCGATATTTCCGGAATCAACGCATCTGTGGTCAATATACAG
AAGGAGATCGATAGGTTGAACGAAGTCGCAAAGAACCTCAACGAGTCATTGATTGACCTTCAG
GAGTTGGGGAAATACGAACAATACATTAAGTGGCCTTGGTATATCTGGCTTGGATTCATCGCA
GGACTTATCGCAATTGTGATGGTCACCATCATGCTGTGTTGCATGACGAGCTGTTGCAGCTGC
CTGAAAGGGTGCTGCTCCTGCGGCTCTTGTTGT
SEQ ID NO: 7
ATGTTCGTCTTCCTGGTGCTGCTGCCTTTGGTTTCATCCCAATGCGTGAACCTCACCACACGT
ACACAGCTGCCGCCGGCCTATACGAACTCATTTACCAGAGGCGTCTACTATCCGGATAAGGTC
TTTCGGAGTAGCGTCCTGCACAGCACCCAGGATCTGTTTCTGCCCTTCTTCTCAAACGTGACA
TGGTTTCACGCGATCCACGTTTCAGGCACTAACGGGACGAAACGATTCGcCAATCCGGTGCTC
CCCTTCAACGACGGCGTGTACTTCGCATCAACCGAAAAGTCCAATATCATTCGTGGTTGGATC
TTCGGGACCACACTTGACAGTAAAACTCAATCTCTGTTGATCGTGAACAATGCCACCAACGTG
GTAATCAAGGTGTGCGAGTTCCAGTTCTGCAACGACCCGTTCCTGGGCGTGTACTATCATAAG
AATAATAAGTCATGGATGGAGAGCGAATTTCGGGTGTACTCATCCGCTAACAACTGTACCTTC
GAGTACGTTTCACAACCGTTCCTCATGGATCTTGAGGGCAAGCAAGGCAACTTTAAGAACCTG
CGGGAGTTCGTATTCAAGAACATCGACGGATACTTCAAAATCTACTCAAAACATACCCCCATA
AACCTCGTAAGGGACCTGCCACAAGGCTTCTCTGCGCTGGAGCCCCTCGTGGACCTGCCTATC
GGCATCAATATTACCCGGTTCCAGACACTGatcTCTTATCTCACCCCGGGAGACAGCTCATCT
GGCTGGACGGCCGGGGCAGCAGCATACTATGTCGGGTACCTGCAGCCACGCACCTTTCTGCTC
AAGTACAACGAGAACGGGACAATCACCGACGCAGTGGATTGCGCCCTGGACCCACTGTCCGAG
ACTAAATGCACCCTCAAGAGCTTTACCGTCGAGAAGGGTATATACCAGACAAGCAATTTCCGC
GTGCAGCCTACTGAGAGTATCGTGCGCTTCCCAAACATCACTAATCTGTGTCCATTCGGAGAG
GTATTCAATGCAACAAGGTTCGCCAGTGTGTACGCGTGGAATCGGAAACGAATTAGTAATTGC
GTGGCAGACTACAGCGTTCTTTACAACAGCGCTAGTTTCTCAACCTTCAAATGCTACGGGGTT
TCCCCGACGAAGTTGAACGACCTGTGTTTCACAAACGTGTACGCTGACAGTTTCGTCATCCGG
GGTGACGAGGTTAGGCAGATAGCCCCCGGACAGACCGGGAACATCGCCGACTACAACTACAAG
CTCCCTGACGACTTCACGGGTTGTGTAATCGCCTGGAATTCTAATAACCTGGACAGCAAAGTG
GGCGGAAACTACAACTATCTTTACCGCCTGTTCCGTAAAAGCAACCTGAAGCCATTCGAACGC
GACATATCTACGGAGATATACCAAGCTGGCTCTACCCCATGCAACGGAGTGaAAGGGTTCAAC
TGCTATTTCCCCCTGCAGAGCTACGGGTTTCAGCCAACCtACGGCGTCGGATATCAGCCCTAT
AGGGTCGTTGTCCTGTCCTTCGAGCTGCTGCACGCCCCGGCGACCGTGTGCGGCCCAAAGAAA
TCAACGAACCTGGTGAAGAATAAGTGCGTTAACTTTAATTTTAACGGCCTCACTGGGACTGGG
GTCTTGACAGAATCCAATAAGAAATTCCTTCCCTTTCAGCAGTTCGGTCGCGATATCGCCGAT
ACAACAGACGCCGTTCGGGACCCCCAAACTTTGGAAATACTCGATATCACTCCCTGCAGTTTC
GGCGGCGTGTCCGTCATCACGCCTGGCACCAACACCAGCAATCAAGTAGCCGTCTTGTACCAA
GgCGTCAATTGTACCGAGGTGCCAGTCGCAATCCACGCCGACCAGCTCACCCCAACGTGGAGA
GTGTACTCAACGGGCAGCAACGTATTCCAGACCAGGGCGGGTTGCCTGATCGGCGCCGAGCAC
GTGAATAATAGCTACGAATGCGATATCCCAATCGGGGCGGGCATTTGTGCCTCATACCAAACC
CAAACAAACTCACCCAGGCGGGCTAGGTCTGTTGCCAGCCAGAGCATAATCGCTTATACCATG
TCCCTGGGCGtTGAGAACAGTGTGGCCTATAGCAACAATTCCATaGCTATTCCTACCAACTTC
ACAATCTCCGTAACTACTGAGATCCTTCCTGTCTCCATGACTAAAACTTCTGTCGATTGCACC
ATGTATATCTGCGGCGACAGCACAGAGTGTTCAAACCTGCTGCTGCAGTACGGTTCCTTCTGC
ACCCAGCTGAATAGGGCGTTGACCGGTATCGCGGTCGAGCAGGATAAGAATACGCAGGAAGTG
TTCGCCCAGGTTAAGCAGATTTATAAGACGCCCCCTATCAAGGACTTCGGCGGATTCAACTTC
TCCCAGATTCTGCCTGACCCCAGCAAGCCTTCTAAAAGATCCTTCATCGAAGATCTATTGTTT
AATAAGGTAACTTTGGCCGACGCCGGGTTTATTAAGCAGTACGGCGACTGTTTGGGCGACATC
GCAGCAAGGGATCTGATCTGCGCCCAGAAATTCAATGGACTGACAGTGCTGCCCCCACTCTTG
ACGGACGAGATGATCGCCCAGTATACCAGCGCGCTCCTGGCCGGCACCATTACCAGTGGATGG
ACATTCGGCGCTGGCGCGGCTCTGCAGATCCCTTTCGCAATGCAGATGGCGTACCGTTTCAAC
GGCATAGGTGTAACCCAAAACGTACTGTACGAAAATCAGAAGCTGATAGCTAATCAGTTCAAC
AGCGCCATCGGGAAGATCCAGGATTCTCTGTCAAGCACTGCCTCAGCCCTCGGGAAGTTGCAG
GACGTAGTTAATCAGAACGCTCAGGCCTTGAATACCCTCGTAAAGCAGTTGTCCAGTAACTTC
GGAGCTATCTCTAGCGTGTTGAACGACATATTGAGCAGGCTCGATAAGGTAGAAGCCGAGGTA
CAGATCGACCGACTGATTACCGGACGACTGCAGTCCCTTCAAACTTACGTAACGCAGCAGCTG
ATCAGGGCCGCCGAGATTAGGGCCAGCGCGAACCTCGCCGCAACCAAGATGAGTGAATGCGTT
CTGGGCCAGAGTAAGCGTGTGGACTTCTGCGGCAAAGGTTACCACCTCATGTCTTTTCCACAA
AGTGCTCCCCACGGGGTGGTATTTCTCCACGTCACGTACGTGCCAGCTCAGGAGAAGAATTTT
ACTACAGCACCAGCTATATGCCACGACGGGAAGGCCCATTTCCCCCGGGAAGGGGTGTTCGTA
AGCAACGGTACTCATTGGTTCGTTACCCAGCGGAACTTCTACGAGCCCCAGATTATCACCACT
GATAATACCTTCGTCAGCGGAAATTGCGACGTGGTCATTGGGATCGTGAATAATACTGTGTAC
GACCCGCTCCAGCCCGAGCTGGATAGCTTTAAAGAAGAACTGGACAAGTACTTCAAGAACCAC
ACCTCTCCGGACGTGGACCTGGGCGATATTTCCGGAATCAACGCATCTGTGGTCAATATACAG
AAGGAGATCGATAGGTTGAACGAAGTCGCAAAGAACCTCAACGAGTCATTGATTGACCTTCAG
GAGTTGGGGAAATACGAACAATACATTAAGTGGCCTTGGTATATCTGGCTTGGATTCATCGCA
GGACTTATCGCAATTGTGATGGTCACCATCATGCTGTGTTGCATGACGAGCTGTTGCAGCTGC
CTGAAAGGGTGCTGCTCCTGCGGCTCTTGTTGT
SEQ ID NO: 8
ATGTTCGTCTTCCTGGTGCTGCTGCCTTTGGTTTCATCCCAATGCGTGAACCTCAgaACACGT
ACACAGCTGCCGCCGGCCTATACGAACTCATTTACCAGAGGCGTCTACTATCCGGATAAGGTC
TTTCGGAGTAGCGTCCTGCACAGCACCCAGGATCTGTTTCTGCCCTTCTTCTCAAACGTGACA
TGGTTTCACGCGATCCACGTTTCAGGCACTAACGGGACGAAACGATTCGACAATCCGGTGCTC
CCCTTCAACGACGGCGTGTACTTCGCATCAACCGAAAAGTCCAATATCATTCGTGGTTGGATC
TTCGGGACCACACTTGACAGTAAAACTCAATCTCTGTTGATCGTGAACAATGCCACCAACGTG
GTAATCAAGGTGTGCGAGTTCCAGTTCTGCAACGACCCGTTCCTGGaCGTGTACTATCATAAG
AATAATAAGTCATGGATGGAGAGCGgAGTGTACTCATCCGCTAACAACTGTACCTTCGAGTAC
GTTTCACAACCGTTCCTCATGGATCTTGAGGGCAAGCAAGGCAACTTTAAGAACCTGCGGGAG
TTCGTATTCAAGAACATCGACGGATACTTCAAAATCTACTCAAAACATACCCCCATAAACCTC
GTAAGGGACCTGCCACAAGGCTTCTCTGCGCTGGAGCCCCTCGTGGACCTGCCTATCGGCATC
AATATTACCCGGTTCCAGACACTGTTGGCCCTGCACCGGTCTTATCTCACCCCGGGAGACAGC
TCATCTGGCTGGACGGCCGGGGCAGCAGCATACTATGTCGGGTACCTGCAGCCACGCACCTTT
CTGCTCAAGTACAACGAGAACGGGACAATCACCGACGCAGTGGATTGCGCCCTGGACCCACTG
TCCGAGACTAAATGCACCCTCAAGAGCTTTACCGTCGAGAAGGGTATATACCAGACAAGCAAT
TTCCGCGTGCAGCCTACTGAGAGTATCGTGCGCTTCCCAAACATCACTAATCTGTGTCCATTC
GGAGAGGTATTCAATGCAACAAGGTTCGCCAGTGTGTACGCGTGGAATCGGAAACGAATTAGT
AATTGCGTGGCAGACTACAGCGTTCTTTACAACAGCGCTAGTTTCTCAACCTTCAAATGCTAC
GGGGTTTCCCCGACGAAGTTGAACGACCTGTGTTTCACAAACGTGTACGCTGACAGTTTCGTC
ATCCGGGGTGACGAGGTTAGGCAGATAGCCCCCGGACAGACCGGGAAAATCGCCGACTACAAC
TACAAGCTCCCTGACGACTTCACGGGTTGTGTAATCGCCTGGAATTCTAATAACCTGGACAGC
AAAGTGGGCGGAAACTACAACTATCgTTACCGCCTGTTCCGTAAAAGCAACCTGAAGCCATTC
GAACGCGACATATCTACGGAGATATACCAAGCTGGCTCTAaaCCATGCAACGGAGTGGAAGGG
TTCAACTGCTATTTCCCCCTGCAGAGCTACGGGTTTCAGCCAACCAACGGCGTCGGATATCAG
CCCTATAGGGTCGTTGTCCTGTCCTTCGAGCTGCTGCACGCCCCGGCGACCGTGTGCGGCCCA
AAGAAATCAACGAACCTGGTGAAGAATAAGTGCGTTAACTTTAATTTTAACGGCCTCACTGGG
ACTGGGGTCTTGACAGAATCCAATAAGAAATTCCTTCCCTTTCAGCAGTTCGGTCGCGATATC
GCCGATACAACAGACGCCGTTCGGGACCCCCAAACTTTGGAAATACTCGATATCACTCCCTGC
AGTTTCGGCGGCGTGTCCGTCATCACGCCTGGCACCAACACCAGCAATCAAGTAGCCGTCTTG
TACCAAGgCGTCAATTGTACCGAGGTGCCAGTCGCAATCCACGCCGACCAGCTCACCCCAACG
TGGAGAGTGTACTCAACGGGCAGCAACGTATTCCAGACCAGGGCGGGTTGCCTGATCGGCGCC
GAGCACGTGAATAATAGCTACGAATGCGATATCCCAATCGGGGCGGGCATTTGTGCCTCATAC
CAAACCCAAACAAACTCACgCAGGCGGGCTAGGTCTGTTGCCAGCCAGAGCATAATCGCTTAT
ACCATGTCCCTGGGCGCTGAGAACAGTGTGGCCTATAGCAACAATTCCATaGCTATTCCTACC
AACTTCACAATCTCCGTAACTACTGAGATCCTTCCTGTCTCCATGACTAAAACTTCTGTCGAT
TGCACCATGTATATCTGCGGCGACAGCACAGAGTGTTCAAACCTGCTGCTGCAGTACGGTTCC
TTCTGCACCCAGCTGAATAGGGCGTTGACCGGTATCGCGGTCGAGCAGGATAAGAATACGCAG
GAAGTGTTCGCCCAGGTTAAGCAGATTTATAAGACGCCCCCTATCAAGGACTTCGGCGGATTC
AACTTCTCCCAGATTCTGCCTGACCCCAGCAAGCCTTCTAAAAGATCCTTCATCGAAGATCTA
TTGTTTAATAAGGTAACTTTGGCCGACGCCGGGTTTATTAAGCAGTACGGCGACTGTTTGGGC
GACATCGCAGCAAGGGATCTGATCTGCGCCCAGAAATTCAATGGACTGACAGTGCTGCCCCCA
CTCTTGACGGACGAGATGATCGCCCAGTATACCAGCGCGCTCCTGGCCGGCACCATTACCAGT
GGATGGACATTCGGCGCTGGCGCGGCTCTGCAGATCCCTTTCGCAATGCAGATGGCGTACCGT
TTCAACGGCATAGGTGTAACCCAAAACGTACTGTACGAAAATCAGAAGCTGATAGCTAATCAG
TTCAACAGCGCCATCGGGAAGATCCAGGATTCTCTGTCAAGCACTGCCTCAGCCCTCGGGAAG
TTGCAGaACGTAGTTAATCAGAACGCTCAGGCCTTGAATACCCTCGTAAAGCAGTTGTCCAGT
AACTTCGGAGCTATCTCTAGCGTGTTGAACGACATATTGAGCAGGCTCGATAAGGTAGAAGCC
GAGGTACAGATCGACCGACTGATTACCGGACGACTGCAGTCCCTTCAAACTTACGTAACGCAG
CAGCTGATCAGGGCCGCCGAGATTAGGGCCAGCGCGAACCTCGCCGCAACCAAGATGAGTGAA
TGCGTTCTGGGCCAGAGTAAGCGTGTGGACTTCTGCGGCAAAGGTTACCACCTCATGTCTTTT
CCACAAAGTGCTCCCCACGGGGTGGTATTTCTCCACGTCACGTACGTGCCAGCTCAGGAGAAG
AATTTTACTACAGCACCAGCTATATGCCACGACGGGAAGGCCCATTTCCCCCGGGAAGGGGTG
TTCGTAAGCAACGGTACTCATTGGTTCGTTACCCAGCGGAACTTCTACGAGCCCCAGATTATC
ACCACTGATAATACCTTCGTCAGCGGAAATTGCGACGTGGTCATTGGGATCGTGAATAATACT
GTGTACGACCCGCTCCAGCCCGAGCTGGATAGCTTTAAAGAAGAACTGGACAAGTACTTCAAG
AACCACACCTCTCCGGACGTGGACCTGGGCGATATTTCCGGAATCAACGCATCTGTGGTCAAT
ATACAGAAGGAGATCGATAGGTTGAACGAAGTCGCAAAGAACCTCAACGAGTCATTGATTGAC
CTTCAGGAGTTGGGGAAATACGAACAATACATTAAGTGGCCTTGGTATATCTGGCTTGGATTC
ATCGCAGGACTTATCGCAATTGTGATGGTCACCATCATGCTGTGTTGCATGACGAGCTGTTGC
AGCTGCCTGAAAGGGTGCTGCTCCTGCGGCTCTTGTTGT

In various embodiments, the nucleic acid includes the nucleic acid encoding the S protein from a variant of SARS-CoV2 virus. For example, the S protein/the full-length S protein is the S protein from an alpha variant, a beta variant, a delta variant, and the like. In some examples, the nucleic acid encoding the S protein variants comprises SEQ ID NO: 6 (alpha variant), SEQ ID NO: 7 (beta variant), and/or SEQ ID NO: 8 (delta variant).

In various embodiments, the nucleic acid comprises a sequence sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity with SEQ ID NO: 2, SEQ ID NO: 6, SEQ ID NO: 7, and/or SEQ ID NO: 8. In some embodiments, the nucleic acid comprises a sequence sharing at least about 99.1%, at least about 99.2%, at least about 99.3%, at least about 99.4%, at least about 99.5%, at least about 99.6%, at least about 99.7%, at least about 99.8% or at least about 99.9% sequence identity with SEQ ID NO: 2, SEQ ID NO: 6, SEQ ID NO: 7, and/or SEQ ID NO: 8.

In various embodiments, the nucleic acid comprises a degenerate sequence of SEQ ID NO. 2, SEQ ID NO: 6, SEQ ID NO: 7, and/or SEQ ID NO: 8.

In various embodiments, the nucleic acid sequence encoding for the S protein/full-length S protein comprises no less than about 4000 nucleotides/bases, no less than about 3950 nucleotides/bases, no less than about 3900 nucleotides/bases, no less than about 3850 nucleotides/bases, no less than about 3800 nucleotides/bases, no less than about 3750 nucleotides/bases, no less than about 3700 nucleotides/bases, no less than about 3650 nucleotides/bases, no less than about 3600 nucleotides/bases, no less than about 3550 nucleotides/bases or no less than about 3500 nucleotides/bases. In various embodiments, the nucleic acid sequence encoding for the S protein/full-length S protein comprises no less than about 3830 nucleotides/bases, no less than about 3829 nucleotides/bases, no less than about 3828 nucleotides/bases, no less than about 3827 nucleotides/bases, no less than about 3826 nucleotides/bases, no less than about 3825 nucleotides/bases, no less than about 3824 nucleotides/bases, no less than about 3823 nucleotides/bases, no less than about 3822 nucleotides/bases, no less than about 3821 nucleotides/bases, no less than about 3820 nucleotides/bases, no less than about 3819 nucleotides/bases, no less than about 3818 nucleotides/bases, no less than about 3817 nucleotides/bases, no less than about 3816 nucleotides/bases, no less than about 3815 nucleotides/bases, no less than about 3814 nucleotides/bases, no less than about 3813 nucleotides/bases, no less than about 3812 nucleotides/bases, no less than about 3811 nucleotides/bases, no less than about 3810 nucleotides/bases, no less than about 3809 nucleotides/bases, no less than about 3808 nucleotides/bases, no less than about 3807 nucleotides/bases, no less than about 3806 nucleotides/bases, no less than about 3805 nucleotides/bases, no less than about 3804 nucleotides/bases, no less than about 3803 nucleotides/bases, no less than about 3802 nucleotides/bases, no less than about 3801 nucleotides/bases or no less than about 3800 nucleotides/bases. In various embodiments, the nucleic acid sequence encoding for the S protein/full-length S protein comprises from about 2000 to about 4000 nucleotides/bases, from about 3000 to about 4000 nucleotides/bases, from about 3200 to about 4000 nucleotides/bases, from about 3400 to about 4000 nucleotides/bases, from about 3600 to about 3900 nucleotides/bases, from about 3700 to about 3900 nucleotides/bases, from about 3800 to about 3850 nucleotides/bases, from about 3820 to about 3830 nucleotides/bases or from about 3820 to about 3825 nucleotides/bases. In one embodiment, the nucleic acid sequence encoding for the S protein/full-length S protein comprises about 3822 nucleotides/bases.

In various embodiments, the nucleic acid sequence encoding for the S protein comprises one or more mutation(s) e.g. deletion(s), insertion(s), and/or substitution(s) with other nucleotide(s)/base(s) as compared to SEQ ID NO: 2, SEQ ID NO: 6, SEQ ID NO: 7, and/or SEQ ID NO: 8. In various embodiments, the mutated nucleic acid sequence encodes for a protein of SEQ ID NO. 1, SEQ ID NO: 3, SEQ ID NO: 4, SEQ ID NO: 5, or a protein sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity with SEQ ID NO: 1, SEQ ID NO: 3, SEQ ID NO: 4, and/or SEQ ID NO: 5. In various embodiments, the mutated nucleic acid sequence encodes for a protein sharing at least about 99.1%, at least about 99.2%, at least about 99.3%, at least about 99.4%, at least about 99.5%, at least about 99.6%, at least about 99.7%, at least about 99.8% or at least about 99.9% sequence identity with SEQ ID NO: 1, SEQ ID NO: 3, SEQ ID NO: 4, and/or SEQ ID NO: 5.

In various embodiments, the mutated nucleic acid sequence encodes for a protein that has substantially the same function, activity, structure, folding and/or antigenic properties as a protein of SEQ ID NO: 1, SEQ ID NO: 3, SEQ ID NO: 4, and/or SEQ ID NO: 5.

In various embodiments, the nucleic acid sequence is codon-optimised.

In various embodiments, the host cell as described herein bound to an antigen binding protein or a fragment thereof, optionally an antibody, further optionally an antibody against SARS-CoV-2.

In another aspect, there is provided a method of detecting an antibody or fragment thereof against SARS-CoV-2 in a subject, the method comprising:
contacting/incubating a sample from the subject with the host cell of the present disclosure; and
detecting a binding of antibody or fragment thereof to the host cell.

In various embodiments, the sample comprises a biological sample. In various embodiments, the biological sample comprises a fluid biological sample or a liquid biological sample. The fluid biological sample or liquid biological sample may be blood, serum, plasma, sputum, lavage fluid, cerebrospinal fluid, urine, semen, sweat, tears, saliva, and the like. In some embodiments, the fluid biological sample or liquid biological sample comprises whole blood, blood serum, blood plasma or processed fractions thereof. In some embodiments, the fluid biological sample comprises blood serum or blood plasma. In some embodiments, the fluid biological sample comprises antigen binding proteins such as antibodies.

In various embodiments, the sample is collected/obtained from the subject about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days or about 30 days post-infection onset (pio).

In various embodiments, the sample is collected/obtained from a subject suspected of having the infection. In various embodiments, the sample is collected/obtained from a subject showing symptoms associated with the infection. In various embodiments, the sample is collected/obtained from an asymptomatic subject.

Embodiments of the method allows for multiplexing, where different isotypes (e.g. IgA, IgG and IgM) or IgG subclasses (e.g. IgG1, IgG2, IgG3 and IgG4) can be detected and/or distinguished in a single test. In various embodiments therefore, the method is capable of detecting at least about one, at least about two, at least about three, at least about four, at least about five or at least about six different antibodies, optionally wherein the antibodies are selected from the group consisting of IgA, IgM, IgG including IgG1, IgG2, IgG3 and IgG4.

In one embodiment, the antibody comprises IgG. Advantageously, detecting IgG1 as compared to total IgG may lead to higher sensitivity.

In one aspect, there is provided a method of identifying/characterizing coronavirus infection, optionally SARS-CoV-2 infection in a subject, the method comprising: contacting/incubating a sample from the subject with an isolated host cell comprising a nucleic acid encoding for the spike protein (S protein) of the coronavirus; and detecting a binding of an antibody or fragment thereof to the host cell.

In various embodiments, there is provided a method of identifying/characterizing coronavirus infection, optionally SARS-CoV-2 infection in a subject, the method comprising: contacting/incubating a sample from the subject with the host cell as described herein; and detecting a binding of the antibody or fragment thereof to the host cell.

In various embodiments, a positive response for at least about one, at least about two, at least about three, at least about four at least about five or at least about six isotypes (e.g. IgM, IgG, and four IgG subclasses) is indicative that the subject has a present or a previous/past coronavirus infection, optionally a present or a previous/past SARS-CoV-2 infection.

In some embodiments, the method comprises a method of stratifying a subject for treatment. For example, a strong antibody response may be indicative of a potentially severe clinical outcome and hence subjects showing a strong antibody response may be closely monitored and/or given early intervention/treatment. For example, given that that asymptomatic subjects are found to have barely any IgG2 and IgG4, a IgG2 and/or IgG4 response may be indicative of a potentially severe clinical outcome and hence subjects showing a IgG2 and/or IgG4 response may be closely monitored and/or given early intervention/treatment.

In various embodiments, the method further comprises a step of treating the subject for coronavirus infection, optionally SARS-CoV-2, when the subject is identified to have a coronavirus infection, optionally SARS-CoV-2. Treating the subject may comprise administering to the subject coronavirus/SARS-CoV-2 therapeutic agents or therapeutic agents capable of alleviating or slowing down the medical condition and/or its symptoms. In various embodiments thereof, there is provided a method of treating coronavirus infection, optionally SARS-CoV-2, in a subject, the method comprising, contacting/incubating a sample from the subject with the host cell of the present disclosure, detecting a binding of the antibody or fragment thereof to the host cell and administering to the subject coronavirus/SARS-CoV-2 therapeutic agents or therapeutic agents capable of alleviating or slowing down the medical condition and/or its symptoms when a binding event is detected.

In yet another aspect, there is provided a method of identifying a subject having immunity for coronavirus infection, optionally SARS-CoV-2 infection, the method comprising: contacting/incubating a sample from the subject with the host cell of the present disclosure; and detecting a binding of the antibody or fragment thereof to the host cell.

In another aspect, there is provided, a method of identifying a subject having immunity for coronavirus infection, optionally SARS-CoV-2 infection, the method comprising: contacting/incubating a sample from the subject with an isolated host cell comprising a nucleic acid encoding for the spike protein (S protein) of the coronavirus; and detecting a binding of an antibody or fragment thereof to the host cell.

Without wishing to be bound by theory, it is believed that IgG1 and IgG3 presence is indicative of a TH1 response, which is a pro-inflammatory response and is particularly important in protective immunity against viruses. Without wishing to be bound by theory, it is believed that IgG2 and IgG4 response is indicative of a TH2 response, which is less effective in mediating protective immunity against viruses. In embodiments of the method, the detection of predominant IgG isotypes allows for the identification of the type of immune response (TH1 or TH2) in the subject. For example, the detection of a predominantly IgG1 and/or IgG3 may be indicative that the subject has immunity against coronavirus infection, optionally SARS-CoV-2 infection.

In yet another aspect, there is provided, a method of assessing the efficiency of a coronavirus vaccine in a subject, optionally SARS-CoV-1 vaccine, the method comprising contacting/incubating a sample from the subject with an isolated host cell comprising a nucleic acid encoding for the spike protein (S protein) of the coronavirus; and detecting a binding of an antibody or fragment thereof to the host cell. In various embodiments, the subject has received at least one dose of a coronavirus vaccine. In various embodiments, the subject has received two, or three, or four, or more doses of a coronavirus vaccine.

In various embodiments, the detecting step comprises using a fluorescence detection instrument such as flow cytometer to detect a binding of antibody or fragment thereof to the host cell.

In various embodiments, the detecting step comprises performing flow cytometry to detect a binding of antibody or fragment thereof to the host cell.

In various embodiments, the method further comprising contacting/incubating the sample with one or more detection or secondary antibody.

In various embodiments, the detection or secondary antibody is selected from the group consisting of: anti-human IgA, anti-human IgG, anti-human IgG1, anti-human IgG2, anti-human IgG3, anti-human Ig4, anti-human IgM, mouse anti-human IgG1, mouse anti-human IgG2, mouse anti-human IgG3 and mouse anti-human IgG4. It will be appreciated that other suitable detection or secondary antibodies may also be used.

In various embodiments, the detection or secondary antibody is coupled to/attached to/conjugated to/labelled with a fluorescent tag or molecule. In various embodiments, the fluorescent tag or molecule comprises a fluorochrome or fluorophore. The fluorescent tags or molecules may be any that are detectable by a fluorescence detection instrument such as a flow cytometer. Examples include, but are not limited to, fluorescein isothiocyanate (FITC), Alexa Fluor 647, Alexa Fluor 488, green fluorescent protein (GFP), carboxyfluorescein succinimidyl ester (CFSE), carboxyfluorescein diacetate succinimidyl ester (CFDA-SE), DyLight 488, phycoerythrin (PE), propidium iodide (PI), peridinin chlorophyll protein complex (PerCP), PerCP-Cy5.5, PE-Alexa Fluor 700, PE-Cy5 (TRI-COLOR), PE-Cy5.5, PE-Alexa Fluor 750, PE-Cy7, allophycocyanin (APC), APC-Cy7, APC-eFluor 780, Alexa Fluor 700, Cy5, Draq-5, Pacific Orange, Amine Aqua, Pacific Blue, 4′,6-diamidino-2-phenylindole HCl (DAPI), Alexa Fluor 405, eFluor 450, eFluor 605 Nanocrystals, eFluor 625 Nanocrystals, and eFluor 650 Nanocrystals.

In various embodiments, the method has high sensitivity and/or high specificity.

In various embodiments, the method has a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or about 100% for SARS-CoV-2 detection, optionally anti-SARS-CoV-2 IgA, IgM and/or IgG (including subclasses) detection. In one example, the method has a sensitivity of about 19% and about 61% for IgM detection at about 5 days pio and about 10 days pio respectively. In one example, the method has a sensitivity of about 86% and about 100% for IgG detection at about 10 days pio and about 23 days pio respectively. In various examples, the method has a sensitivity of about 64%, about 37%, about 46% and about 32% for IgG1, IgG2, IgG3 and IgG4 detection respectively at about 10 days pio. In various examples, the method has a sensitivity of about 100%, about 74%, about 94% and about 67% for IgG1, IgG2, IgG3 and IgG4 detection respectively at about 23 days pio. In various embodiments, the method shows a higher sensitivity for IgG detection than IgM detection. In various embodiments, the method shows a higher sensitivity for IgG1 and/or IgG3 detection as compared to IgG2 and/or IgG4 detection.

In various embodiments, the method has a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or about 100% for SARS-CoV-2 detection, optionally anti-SARS-CoV-2 IgA, IgM and/or IgG detection. In various embodiments, the method shows little or no cross-reactivity with antibodies from one or more of a sample obtained from a subject previously infected with severe acute respiratory syndrome (SARS), a control/healthy sample and a sample obtained from a seasonal human CoV-infected subject. In one example, specific IgM is not detected in the control samples. In one example, the specificity of the method is about 100% for IgM detection. In one example, the specificity of the method is about 94% for IgG detection. In various embodiments, the method shows higher specificity for IgM detection as compared to IgG detection. In various examples, the specificity of the method is about 97%, about 98%, about 98% and about 98% for IgG1, IgG2, IgG3 and IgG4 respectively. In various embodiments, the method shows higher specificity for IgG2, IgG3 and/or IgG4 detection as compared to IgG1 detection.

In various embodiments, the method has higher sensitivity and/or specificity as compared to an ELISA-based method.

In various embodiments, the method is capable of detecting SARS-CoV-2 in a presymptomatic infection, an asymptomatic infection and/or infection with mild symptoms. In various embodiments, the method has high sensitivity for presymptomatic infection, an asymptomatic infection and/or infection with mild symptoms. In various embodiments, the method is capable of detecting at least about 90%, at least about 91%, at least about 92%, at least about 93%, at least about 94%, at least about 95%, at least about 96%, at least about 97%, at least about 98%, at least about 99% or about 100% of presymptomatic infection, an asymptomatic infection and/or infection with mild symptoms. In one example, the method is capable of detecting 97% of asymptomatic infections.

In various examples, the method has higher sensitivity and/or specificity that a method that detects neutralizing antibodies against the receptor-binding domain (RBD) of the S protein.

In various embodiments, the method is a diagnostic method, optionally a serological diagnostic method.

In various embodiments, the method further comprising performing a further assay such as a polymerase chain reaction (PCR)-based assay to detect/confirm coronavirus infection, optionally SARS-CoV-2 infection. In various embodiments, the method is an in-vitro or an ex-vivo method.

In some embodiments, the method may be an in-vivo method.

In yet another aspect, there is provided a vaccine comprising S protein/full-length S protein (e.g. S protein/full-length S protein as described hereinabove) and/or nucleic acids encoding for the S protein/full-length S protein (e.g. a nucleic acid as described hereinabove).

In various embodiments, the vaccine is devoid of or does not comprise or lacks one of more of the other genes/sequences/proteins (other than the gene/sequence encoding for S protein or S protein) of SARS-CoV-2. In some embodiments, the vaccine is devoid of or does not comprise or lacks one of more of the other genes/sequences/proteins of SARS-CoV-2 necessary for replication, transcription and infectious virus assembly. In some embodiments, the vaccine is devoid of or does not comprise or lacks one of more of the other genes/sequences/proteins of small envelope (E) glycoprotein, membrane (M) glycoprotein and nucleocapsid (N) protein, the other accessory proteins and the other non-structural proteins including nsp1 through nsp16.

In various embodiments, there is provided methods of detecting anti-spike protein antibodies comprising the following steps: (a) incubating human biological samples obtained from patients with HEK293T cells transduced with lentiviral particles to stably express the S protein of SARS-CoV2 on the cell surface and (b) analysing said samples using Fluorescence-Activated Cell Sorting.

Also disclosed is a product or a method as described herein.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

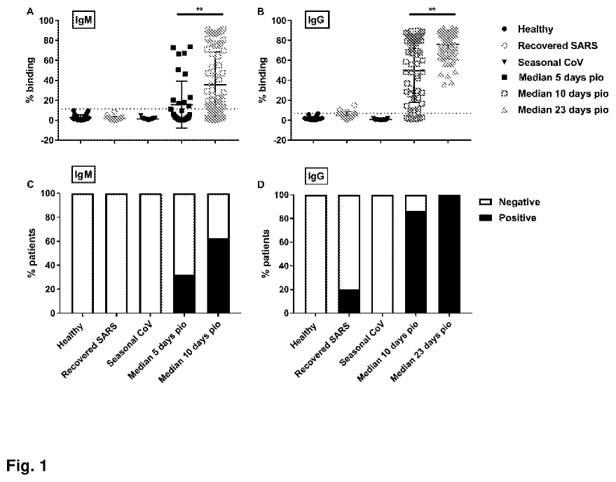

FIG. 1 Specific antibodies against full length S protein. Plasma samples were collected from COVID-19 patients (n=81) at time points with a median of 5 days post-illness onset (pio, n=34), 10 days pio (n=59) and 23 days pio (n=66). Samples were screened at 1:100 dilution for specific (A) IgM and (B) IgG against full length SARS-CoV2 S protein expressed on the surface of HEK293T cells. Control samples include plasma samples from recovered SARS patients (recovered SARS, n=20), plasma samples from healthy donors (Healthy, n=22) and sera from seasonal human CoV-infected patients (Seasonal CoV, n=20). Proportion of individuals having a positive (C) IgM and (D) IgG response were analysed. Data are shown as mean±SD of two independent experiments, with dotted lines indicating mean+3SD of healthy donors. A sample is defined as positive when the binding is more than mean+3SD of the healthy controls. Statistical analysis was carried out using Kruskal-Wallis tests, followed by post hoc Dunn's multiple comparison tests. P-values for comparisons between the different time points are shown, where * indicates P≤1.05, ** indicates P≤1.01.

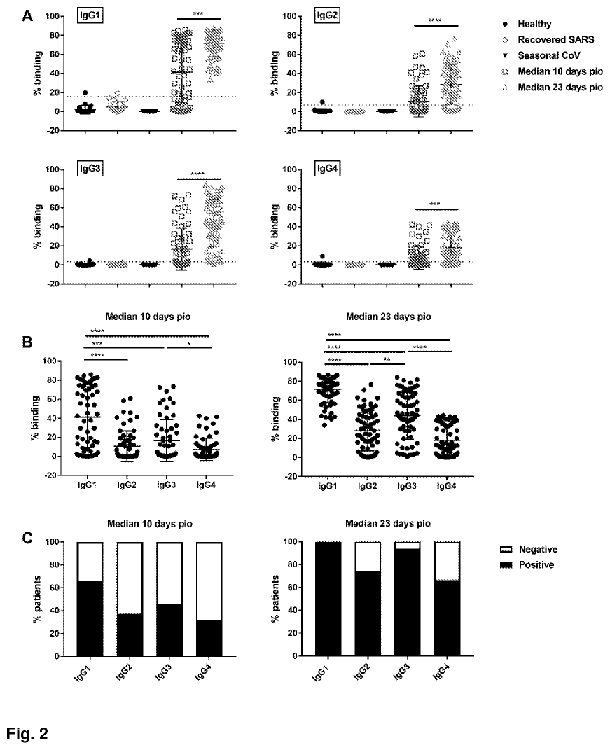

FIG. 2. Specific IgG subclasses against full length S protein. Plasma samples collected from 81 COVID-19 patients at time points with a median of 10 days pio (n=59) and 23 days pio (n=66) were further screened for IgG subclasses, (A) IgG1, (B) IgG2, (C) IgG3, and (D) IgG4. The four IgG subclasses response at median of (E) 10 days pio and (F) 23 days pio were plotted. Proportion of individuals having a positive response at time point (G) median 10 days pio and (H) median 23 days pio were analysed. Data are shown as mean±SD of two independent experiments, with dotted lines indicating mean +3SD of healthy donors. A sample is defined as positive when the binding is more than mean+3SD of the healthy controls. Statistical analysis was carried out using Kruskal-Wallis tests, followed by post hoc Dunn's multiple comparison tests. For (A-D), only P-values for comparisons between the different time points are shown, where * indicates P≤1.05, ** indicates P≤1.01, *** indicates P≤1.001, **** indicates P≤1.0001.

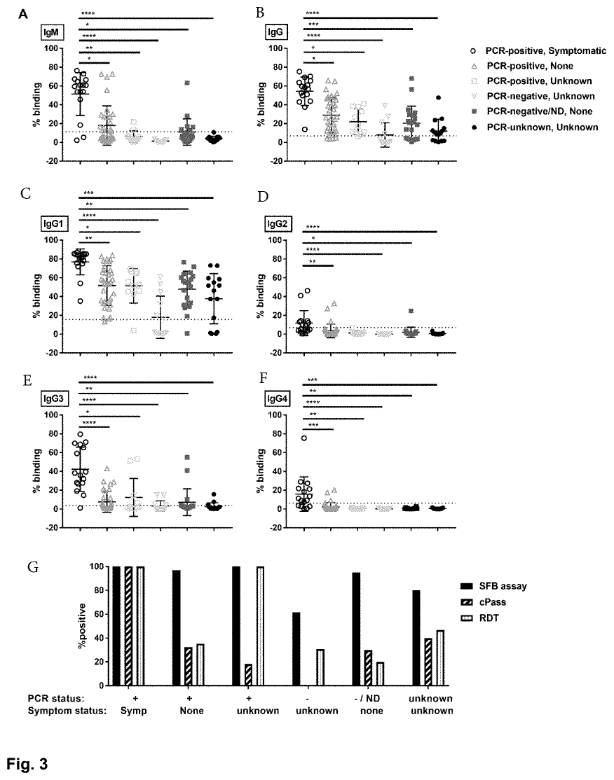

FIG. 3. S protein-specific antibody profile in pre-/asymptomatic infections. Plasma samples were collected from either suspected COVID-19 infections or randomly from the general population under the Singapore Infectious Disease Act. Samples were screened at 1:100 dilution for specific (A) IgM, (B) IgG, (C) IgG1, (D) IgG2, (E) IgG3, (F) IgG4 against full length SARS-CoV2 S protein expressed on the surface of HEK293T cells. A total of 109 samples were analysed and included sera from PCR-positive and symptomatic infections (PCR-positive, symptomatic; n=16), sera from PCR-positive and no symptom infections (PCR-positive, none; n=34), sera from PCR-positive and unknown symptom status infections (PCR-positive, unknown; n=11), sera from PCR-negative and unknown symptom status infections (PCR-negative, unknown; n=13), sera from PCR-negative/not done and no symptom infections (PCR-negative/ND, none; n=20), and sera from PCR-unknown and unknown symptom status infections (PCR-unknown, unknown; n=15). Statistical analysis was carried out using Kruskal-Wallis tests, followed by post hoc Dunn's multiple comparison tests. P-values for comparisons between PCR-positive, symptomatic and PCR-positive and no symptom infections are shown, where * indicates P≤1.05, ** indicates P≤1.01, *** indicates P≤1.001, **** indicates P≤1.0001. (G) Proportion of samples that were positive by the serological tests: SFB IgM/IgG, SFB all isotypes, cPass and RDT. A sample is defined as positive by SFB assay when the binding is more than mean+3SD of the healthy controls. Samples that have a positive IgM and/or IgG were defined as positive by the SFB IgM/IgG assay. Samples that have a positive IgM, IgG, IgG1, IgG2, IgG3 and/or IgG4 were defined as positive by the all isotypes SFB assay. Data are shown as mean±SD of two independent experiments, with dotted lines indicating mean+3SD of healthy donors. +, positive; −, negative; Symp, symptomatic; None, no symptom, ND, not done.

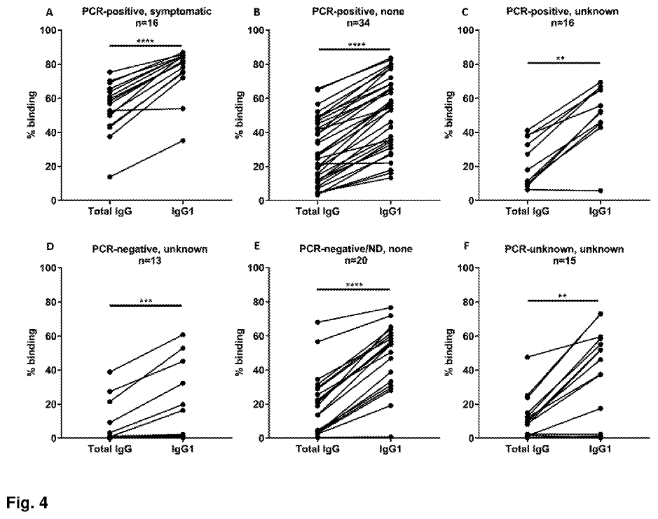

FIG. 4. Comparison of total IgG and IgG1 response. The total IgG response was compared with the IgG1 response for all 109 samples from NPHL: (A) PCR-positive and symptomatic infections (PCR-positive, symptomatic; n=16), (B) PCR-positive and no symptom infections (PCR-positive, none; n=34), (C) PCR-positive and unknown symptom status infections (PCR-positive, unknown; n=11), (D) PCR-negative and unknown symptom status infections (PCR-negative, unknown; n=13), (E) PCR-negative/not done and no symptom infections (PCR-negative/ND, none; n=20), (F) PCR-unknown and unknown symptom status infections (PCR-unknown, unknown; n=15). Statistical analysis was carried out using Wilcoxon matched-pairs signed rank test. P-values for comparisons between the total IgG and IgG1 responses are shown, where * indicates P≤1.05, ** indicates P≤1.01, *** indicates P≤1.001, **** indicates P≤1.0001. Data are shown as mean±SD of two independent experiments, with dotted lines indicating mean+3SD of healthy donors. ND, not done.

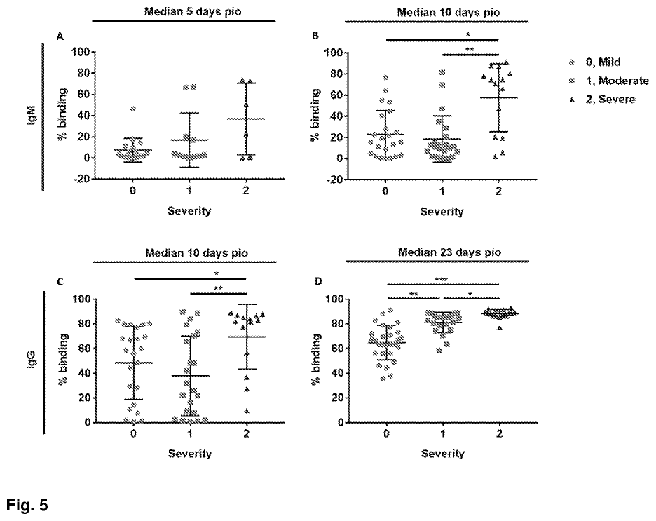

FIG. 5. Association of specific antibodies against full length S protein with disease severity. (A) IgM at median 5 days pio. Samples were classified based on severity denoting mild (clinical severity 0; n=17), moderate (clinical severity 1; n=11), and severe (clinical severity 2; n=6). (B) IgM and (C) IgG at median 10 days pio. Samples were classified based on severity denoting mild (clinical severity 0; n=23), moderate (clinical severity 1; n=12), and severe (clinical severity 2; n=14). (D) IgG at median 23 days pio. Samples were classified based on severity denoting mild (clinical severity 0; n=28), moderate (clinical severity 1; n=22), and severe (clinical severity 2; n=16). Data are presented as mean±SD of two independent experiments. Statistical analysis was carried out using Kruskal-Wallis tests, followed by post hoc Dunn's multiple comparison tests. P-values for comparisons between the different time points are shown, where * indicates P≤1.05, ** indicates P≤1.01, P≤1.001, *** indicates P≤1.001.

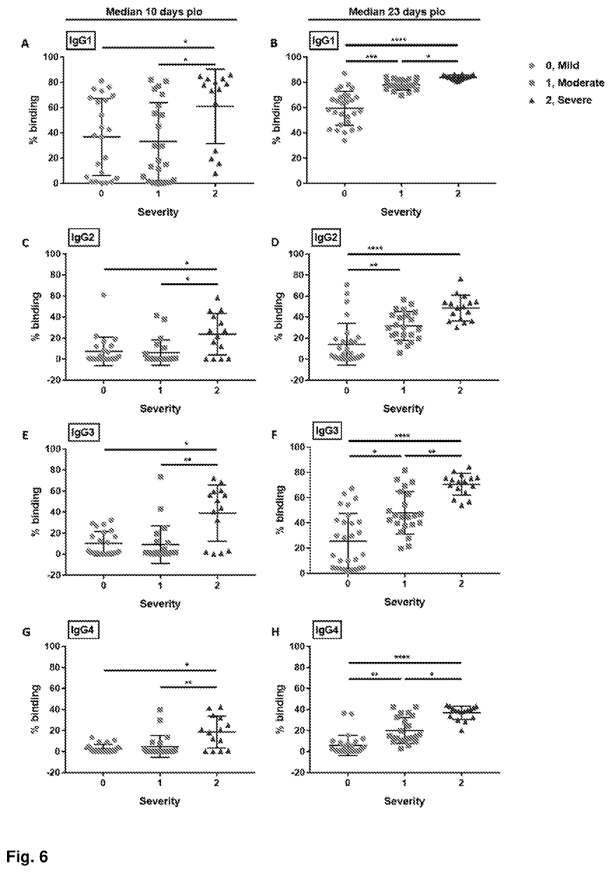

FIG. 6. Association of specific IgG subclasses against full length S protein with disease severity. (A) IgG1, (C) IgG2, (E) IgG3, and (G) IgG4 at median 10 days pio. Samples were classified based on severity denoting mild (clinical severity 0; n=23), moderate (clinical severity 1; n=12), and severe (clinical severity 2; n=14). (B) IgG1, (D) IgG2, (F) IgG3, and (H) IgG4 at median 23 days pio. Samples were classified based on severity denoting mild (clinical severity 0; n=28), moderate (clinical severity 1; n=22), and severe (clinical severity 2; n=16). Data are presented as mean±SD of two independent experiments. Statistical analysis was carried out using Kruskal-Wallis tests, followed by post hoc Dunn's multiple comparison tests. P-values for comparisons between the different time points are shown, where * indicates P≤1.05, ** indicates P≤1.01, *** indicates P≤1.001, **** indicates P≤1.0001.

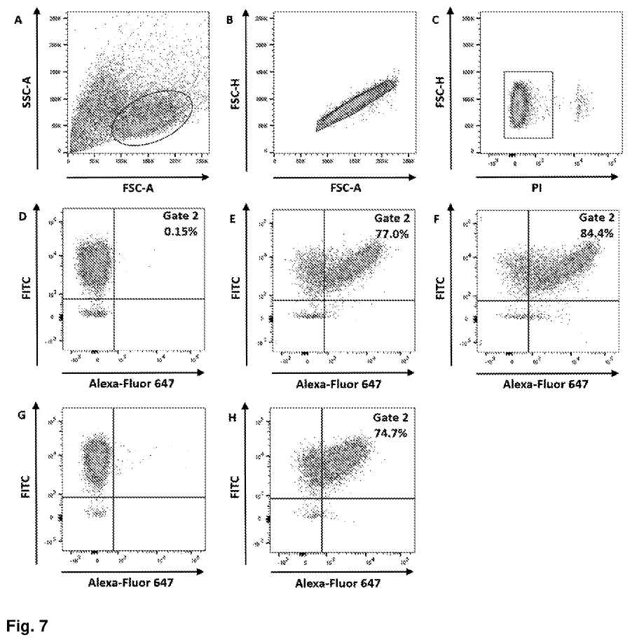

FIG. 7. FACS plot analysis and plasmid map of pHIV-SARS-CoV-2-SP-eGPF. Cells were gated on: (A) FSC-A/SSC-A to exclude cell debris, (B) FSC-A/FSC-H to select for single cells, (C) FSC-A/PI to select for live cells (PI-negative population), (D, E, F, G, H) FITC/Alexa Fluor 647. Binding is determined by the percentage of GFP-positive S protein-expressing cells that are bound by specific antibody, indicated by the events that are Alexa Fluor 647- and FITC-positive (Gate 2). (D) PBS control; (E) 6.25 μg/ml ACE-Human Fc; (F) 1 μg/ml 5A6 S protein RBD-specific monoclonal antibody; (G) healthy control plasma, 1:100 diluted; (H) COVID-19 patient plasma, 1:100 diluted.

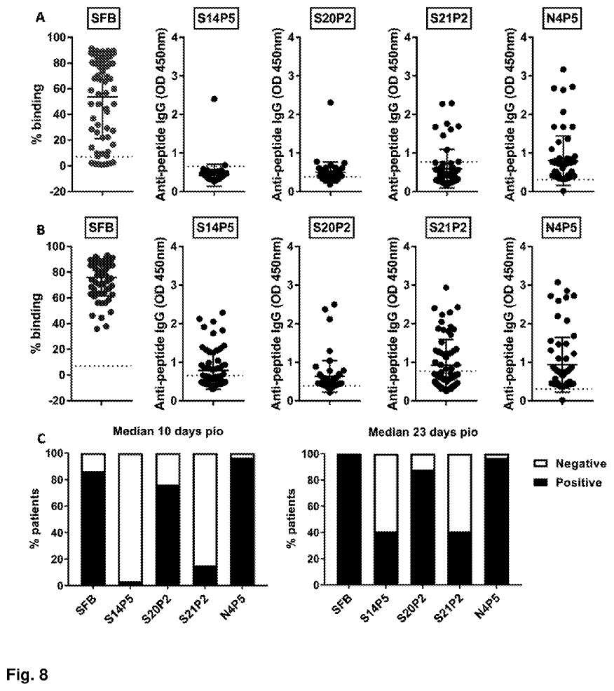

FIG. 8. Comparisons of total IgG response by SFB assay and linear epitope ELISA. Plasma samples were collected from COVID-19 patients (n=81) at time points with a median of 10 days pio (n=59) and 23 days pio (n=66). Total IgG and IgG1 responses at time point (A) median 10 days post-illness onset (pio) and (B) median 23 days pio were compared. Proportion of individuals having a positive response at time point (C) median 10 days pio and (D) median 23 days pio analysed by SFB assay and linear epitope ELISA. Data are shown as mean±SD of two independent experiments, with dotted lines indicating mean+3SD of healthy donors. A sample is defined as positive when the binding is more than mean+3SD of the healthy controls (n=22).

FIG. 9. Association of specific IgM (A-C) and IgG (D-F) against full length S protein with different clinical outcomes, pneumonia, oxygen supple requirement, and intensive care unit (ICU) admission. Negative and positive observations of (A, D) pneumonia, (B, E) oxygen supple requirement, and (C, F) ICU admission are denoted as − and +, respectively. Data are presented as mean±SD of two independent experiments. Statistical analysis was carried out using Kruskal-Wallis tests, followed by post hoc Dunn's multiple comparison tests. P-values for comparisons between the different time points are shown, where * indicates P≤1.05, ** indicates P≤1.01, *** indicates P≤1.001, **** indicates P≤1.0001.

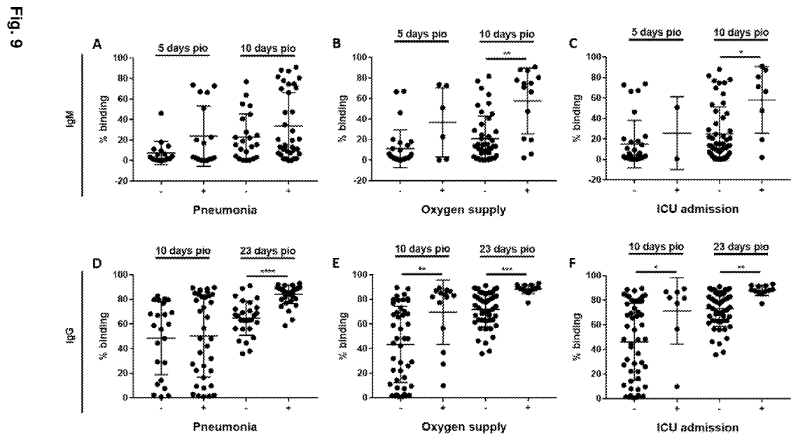

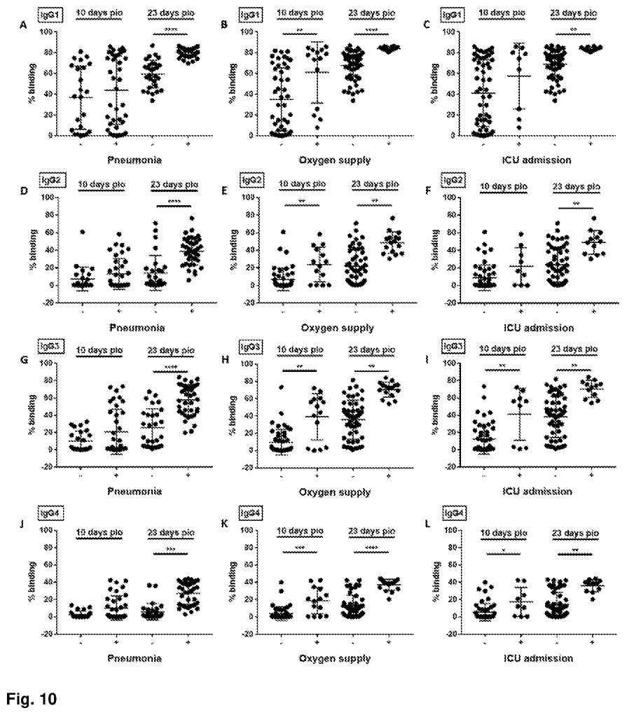

FIG. 10. Association of specific (A-C) IgG1, (D-F) IgG2, (G-I) IgG3, and (J-L) IgG4 against full length S protein with different clinical outcomes, pneumonia, oxygen supple requirement, and intensive care unit (ICU) admission. Negative and positive observations of (A, D, G, J) pneumonia, (B, E, H, K) oxygen supple requirement, and (C, F, I, L) ICU admission are denoted as − and +, respectively. Data are presented as mean±SD of two independent experiments. Statistical analysis was carried out using Kruskal-Wallis tests, followed by post hoc Dunn's multiple comparison tests. P-values for comparisons between the different time points are shown, where * indicates P≤1.05, ** indicates P≤1.01, *** indicates P≤1.001, **** indicates P≤1.0001.

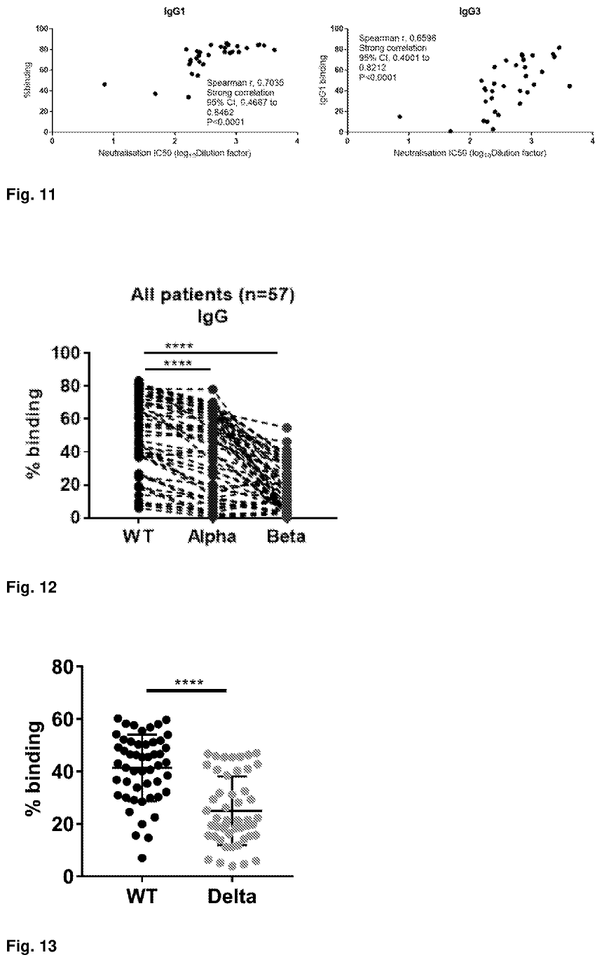

FIG. 11. Correlation between IgG1 and IgG3 subclasses and neutralisation capacity. Spearman r of 0.6 to 0.8 indicates strong correlation.

FIG. 12. S protein-specific antibody profile against wildtype (WT), Alpha and Beta variants. Plasma samples were collected from COVID-19 patients with SARS-CoV-2 infections (n=57) at median 31 days post-illness onset (pio). Samples were screened at 1:100 dilution for specific IgG against the different variants of full length SARS-CoV-2 S protein expressed on the surface of HEK293T cells. Data are shown as mean±SD of two independent experiments. Statistical analysis was carried out on the paired samples using Friedman test, followed by post hoc Dunn's multiple comparison tests. P-values for comparisons between the groups are shown, where **** indicates P≤1.0001.

FIG. 13. S protein-specific antibody profile against wildtype (WT), and delta variants, following COVID-19 vaccination. Plasma samples were collected from vaccinated individuals 70 days post second dose of Pfizer COVID-19 vaccines (n=50).

Samples were screened at 1:100 dilution for specific IgG against the different variants of full length SARS-CoV-2 S protein expressed on the surface of HEK293T cells. Data are shown as mean±SD of two independent experiments. Statistical analysis was carried out on student's t test. P-values for comparisons between the groups are shown, where **** indicates P≤1.0001.

### EXPERIMENTAL SECTION

### Example 1

Experimental Model and Subject Details

Ethics. The study design and protocols for COVID-19, recovered SARS and seasonal human CoV patient cohorts were approved by National Healthcare Group (NHG) Domain Specific Review Board (DSRB) and performed, following ethical guidelines in the approved studies 2012/00917, 2020/00091 and 2020/00076 respectively. Healthy donor samples were collected in accordance with approved studies 2017/2806 and NUS IRB 04-140. Written informed consent was obtained from participants in accordance with the Declaration of Helsinki for Human Research.

All samples received at the National Public Health Laboratory (NPHL) were collected under Singapore Infectious Diseases Act, which allows epidemiological studies and use of data for analysis to control outbreaks (Singapore Statutes Online, 2003).

Plasma Samples

COVID-19 Patients

A total of 81 patients, who were tested PCR-positive for SARS-CoV-2 in the nasopharyngeal swab, were recruited into the study from January to March 2020 (Pung et al., 2020). Demographic data, clinical and laboratory parameters, and clinical severity during the hospitalisation period were retrieved from patient records (Table 2) (Amrun et al., 2020). Patients were classified into three groups based on clinical severity: mild (no pneumonia; clinical severity 0), moderate (pneumonia without hypoxia; clinical severity 1), and severe (pneumonia with hypoxia; clinical severity 2). Whole blood of patients was collected into BD Vacutainer® CPT™ tubes, and centrifuged at 1700 g for 20 min to obtain plasma fractions. Plasma samples were categorised according to three time points: median 5 days post-illness onset (pio), median 10 days pio, and median 23 days pio.

Recovered SARS and Seasonal Human CoV Patients

A total of 20 individuals previously diagnosed with SARS-CoV (Table 2) during the outbreak in 2003 (Leong et al., 2006) were contacted and enrolled. Plasma fractions were isolated from recovered SARS individuals described above. Archived samples from human CoV patients (Table 2) collected between 2012-2013 were also used in this study. This included post-infected samples from seven alpha-CoV (229E/NL63) and six beta-CoV (0043) infections confirmed using the SeeGene RV12 respiratory multiplex kit (Jiang et al., 2017).

Samples Received at NPHL

Plasma samples received at the National Public Health Laboratory (NPHL) were collected from convalescent cases, suspected infections and general populations for sero-prevalence studies. A total of 109 samples was categorised based on PCR-status and patient symptoms status (Table 2): (1) PCR-positive and symptomatic, n=16, (2) PCR-positive and pre-/asymptomatic, n=34, (3) PCR-positive and patient symptom status-unknown, n=11, (4) PCR-negative and patient symptom status-unknown, n=13, (5) PCR status-negative/not done and no symptom, n=20, (6) PCR status-unknown and patient symptom status-unknown, n=15.

Method Details

Generation of S Protein-Expressing Cell Line

The SARS-Cov-2 S gene (GenBank: QHD43416.1), which encodes for the S protein, was codon-optimised (Table 3) for expression by human mammalian cells. Full length S gene was cloned into pHIV-eGFP transfer plasmid, via the Xbal and BamHl sites, upstream of an IRES (internal ribosome entry site) and a eGFP gene (Table 3). The transfer plasmid, pHIV-SARS-CoV-2-SP-eGPF, was then co-transfected with the packaging and envelope plasmids (pMD2.G, pMDLg/pRRE and pRSV-Rev) into HEK 293T cells using EndoFectin Lenti. The medium (DMEM+10% FBS) was changed 8-16 h later and the lentiviral particles in the supernatant were collected after a further 48 h incubation. Cells were transduced by adding the lentiviral supernatant and 8 μg/ml polybrene, then centrifuging at 1200×g for 1 h at room temperature. The medium was changed after 8-16 h in the cell culture incubator. After a further 48 h incubation, eGFP-expressing HEK293T cells were sorted, expanded and cryopreserved.

Expression of S protein was confirmed by ACE2 binding (FIG. 8). Briefly, cells were seeded at 1.5×10⁵ cells per well in 96 well V-bottomed plates. The cells were first incubated with ACE2-HuFc (ACE2 protein tagged with a human Fc region, 6.25 μg/ml) before a secondary incubation with a double stain, consisting of Alexa Fluor 647-conjugated anti-human IgG (diluted 1:500) and propidium iodide (PI; diluted 1:2500). Cells were read on LSR4 laser (BD Biosciences) and analyzed using FlowJo (Tree Star).

TABLE 3
DNA sequence of codon-optimised SARS-Cov-2 S gene and primers
used to sequence full length SARS-Cov-2-S protein

| SARS- | ATGTTTGTATTCTTGGTACTTCTCCCATTGGTATCTTCTCAATGCGTTAAC | |
|---|---|---|
| CoV-2 S | CTTACCACACGCACCCAACTGCCCCCGGCCTACACTAATAGCTTTACGC | |
| gene | GGGGTGTCTACTATCCCGACAAAGTCTTTCGATCCAGTGTGCTCCACTC | |
| Codon- | CACCCAGGATCTTTTCCTTCCCTTTTTTTCTAATGTTACGTGGTTCCACG | |
| optimised | CAATCCATGTATCCGGTACGAATGGGACAAAACGCTTTGACAATCCAGT | |
| | GCTGCCATTTAATGATGGAGTGTACTTTGCATCTACCGAGAAGAGTAACA | |
| | TCATCAGAGGATGGATCTTCGGAACGACCTTGGACTCCAAAACGCAATC | |
| | CTTGCTTATCGTTAACAATGCAACGAATGTTGTCATCAAAGTTTGCGAAT | |
| | TCCAATTCTGTAACGATCCCTTCCTCGGTGTTTATTATCATAAAAATAATA | |
| | AATCTTGGATGGAAAGTGAGTTCCGCGTATACAGTTCCGCCAATAATTGT | |
| | ACCTTCGAATACGTAAGTCAACCGTTCTTGATGGATCTGGAAGGTAAACA | |
| | GGGTAACTTTAAGAACCTTCGGGAGTTTGTTTTTAAGAACATAGACGGCT | |
| | ACTTTAAGATCTATAGTAAACATACGCCAATTAACTTGGTTAGAGATCTCC | |
| | CGCAGGGGTTTTCAGCATTGGAGCCGCTCGTCGACCTCCCCATAGGTAT | |
| | AAATATAACTCGGTTTCAAACACTGCTGGCGCTCCACCGCAGCTACCTG | |
| | ACGCCTGGGGATTCTTCTTCCGGTTGGACTGCAGGCGCTGCTGCATATT | |
| | ATGTAGGGTACCTGCAACCGAGAACCTTTCTCCTTAAGTACAACGAGAAT | |
| | GGCACTATTACGGACGCTGTCGATTGTGCACTCGACCCCTTGAGTGAGA | |
| | CGAAGTGTACACTGAAAAGCTTTACTGTTGAAAAGGGAATATATCAGACA | |
| | TCCAACTTTAGAGTTCAGCCAACAGAATCCATCGTTCGATTTCCCAATAT | |
| | TACAAATCTCTGTCCGTTCGGAGAGGTCTTTAATGCTACCCGATTCGCGT | |
| | CAGTATACGCCTGGAACAGAAAGAGAATTTCTAACTGTGTTGCAGATTAT | |
| | AGTGTCCTGTATAATTCTGCGTCTTTTAGCACTTTTAAGTGCTACGGCGT | |
| | TAGCCCCACTAAGTTGAACGACCTTTGTTTCACTAACGTGTATGCCGACT | |
| | CATTCGTCATAAGAGGCGACGAAGTTAGACAAATTGCACCGGGCCAGAC | |
| | GGGAAAGATTGCGGACTACAACTATAAATTGCCTGACGACTTTACAGGA | |
| | TGTGTCATCGCCTGGAATAGTAATAACCTTGACTCCAAAGTCGGTGGCA | |
| | ATTACAATTACTTGTACCGGCTGTTCAGGAAGTCTAATCTCAAACCTTTT | |
| | GAGCGAGATATCAGCACGGAAATTTATCAAGCTGGTAGCACTCCATGTA | |
| | ACGGGGTTGAGGGTTTTAATTGTTATTTTCCATTGCAATCATATGGATTC | |
| | CAACCGACTAACGGTGTTGGGTATCAACCATACAGAGTGGTGGTTTTGT | |
| | CATTTGAACTTCTGCATGCCCCTGCAACAGTGTGCGGACCGAAGAAGAG | |
| | TACGAACCTTGTAAAGAACAAGTGCGTCAACTTCAACTTTAATGGTCTGA | |
| | CGGGTACCGGCGTTCTGACGGAATCCAATAAAAAGTTCTTGCCCTTTCA | |
| | GCAGTTCGGGCGAGATATCGCCGACACTACTGATGCGGTGCGAGATCC | |
| | TCAGACACTTGAGATCCTCGATATTACCCCATGTAGTTTTGGTGGTGTGT | |
| | CTGTGATTACACCCGGCACCAATACGTCAAATCAGGTCGCAGTCTTGTA | |
| | CCAAGACGTGAACTGCACCGAAGTTCCTGTAGCCATTCACGCTGATCAA | |
| | TTGACACCGACATGGAGGGTGTACTCCACCGGATCTAACGTGTTCCAGA | |
| | CCCGCGCGGGGTGTCTTATCGGCGCAGAACATGTGAACAACTCTTACGA | |
| | ATGTGATATTCCTATCGGTGCAGGCATCTGTGCCTCATACCAGACACAAA | |
| | CGAACTCACCAAGGAGGGCAAGGTCAGTAGCCTCACAAAGCATAATAGC | |
| | CTATACGATGAGTCTTGGTGCGGAGAACTCTGTGGCGTACTCTAATAAC | |
| | TCTATCGCCATACCGACTAACTTCACCATTTCTGTTACGACCGAGATCCT | |
| | CCCAGTTTCCATGACTAAGACAAGTGTGGATTGTACAATGTACATCTGCG | |
| | GCGACAGTACTGAGTGCAGTAACCTGCTTCTGCAGTACGGGTCCTTCTG | |
| | CACACAACTTAACCGGGCGCTGACTGGTATAGCGGTTGAACAAGACAAG | |
| | AACACTCAAGAGGTCTTCGCACAAGTAAAACAAATATACAAAACACCACC | |
| | TATTAAAGATTTCGGCGGGTTTAATTTTAGCCAAATCCTTCCAGACCCCA | |
| | GCAAACCCTCTAAGCGCAGCTTCATTGAGGATCTGCTGTTTAACAAGGT | |
| | CACCCTGGCAGACGCGGGCTTTATCAAGCAATACGGTGACTGCCTGGG | |
| | GGATATCGCGGCTCGAGACCTTATATGTGCGCAAAAATTTAATGGACTTA | |
| | CCGTACTTCCTCCATTGCTGACTGACGAGATGATAGCACAGTATACATCT | |
| | GCACTGCTCGCCGGTACAATTACATCAGGGTGGACATTTGGGGCGGGA | |
| | GCTGCGCTCCAGATACCGTTCGCGATGCAGATGGCGTATAGGTTTAATG | |
| | GAATTGGTGTCACGCAAAACGTTCTCTATGAAAACCAGAAGCTGATAGC | |
| | AAATCAGTTCAATTCCGCGATTGGTAAGATACAAGATTCATTGTCTAGTA | |
| | CGGCCTCTGCACTCGGAAAACTCCAAGATGTAGTGAACCAAAACGCCCA | |
| | AGCCCTGAATACACTCGTAAAACAGCTCTCTAGTAATTTTGGGGCCATTT | |
| | CCTCCGTATTGAACGACATCTTGAGTCGCTTGGATAAGGTAGAAGCAGA | |
| | AGTACAAATTGACCGGTTGATCACGGGCAGACTTCAATCACTTCAGACTT | |
| | ATGTTACTCAGCAGCTTATACGAGCTGCAGAAATTCGCGCCTCTGCGAA | |
| | CCTGGCCGCCACTAAAATGTCAGAATGTGTACTGGGACAGAGCAAACGG | |
| | GTGGATTTCTGCGGAAAGGGCTATCATCTGATGAGTTTTCCCCAGTCTG | |
| | CGCCTCATGGTGTAGTATTTCTTCATGTCACATATGTACCAGCCCAAGAA | |
| | AAAAATTTCACAACGGCGCCCGCGATTTGCCATGACGGTAAGGCGCATT | |
| | TTCCTCGCGAGGGCGTTTTCGTGTCTAACGGTACTCACTGGTTCGTAAC | |
| | ACAGCGAAACTTTTACGAGCCTCAGATAATCACGACGGATAACACATTTG | |
| | TCTCCGGCAACTGCGATGTGGTCATCGGTATAGTGAACAATACGGTATA | |
| | TGATCCGCTGCAGCCAGAGCTCGACAGTTTCAAGGAGGAGCTTGACAAA | |
| | TACTTTAAGAACCATACCTCCCCAGACGTAGACCTCGGAGACATATCTG | |
| | GTATCAATGCCTCCGTGGTTAACATACAAAAGGAGATAGATAGACTGAAT | |
| | GAGGTGGCGAAGAATCTGAATGAGTCTCTCATAGATCTGCAGGAACTCG | |
| | GTAAATATGAACAATACATCAAGTGGCCTTGGTACATCTGGCTGGGGTT | |
| | CATAGCGGGCCTGATCGCGATCGTGATGGTAACTATAATGTTGTGTTGC | |
| | ATGACCTCCTGCTGCTCATGCCTTAAAGGTTGTTGTTCTTGCGGGAGCT | |
| | GCTGCAAGTTCGATGAGGATGATTCAGAACCCGTCTTGAAGGGCGTAAA | |
| | ACTTCACTATACGTAA (SEQ ID NO: 2) | |
| Primers | EF1aFor | GGATCTTGGTTCATTCTCAAG (SEQ ID NO: 9) |
| used to | SPseqF1 | GTACCTGCAACCGAGAAC (SEQ ID NO: 10) |
| sequence | SPseqF2 | GGCGTTCTGACGGAATC (SEQ ID NO: 11) |
| full length | SPseqF3 | GCAATACGGTGACTGCC (SEQ ID NO: 12) |
| SARS- | SPseqF4 | CGTGTCTAACGGTACTCAC (SEQ ID NO: 13) |
| CoV-2-S | SPseqR1 | GTTCTCGGTTGCAGGTAC (SEQ ID NO: 14) |
| protein | IRESrev | CATATAGACAAACGCACACC (SEQ ID NO: 15) |

Flow Cytometry Assay for S Protein Antibody Detection

S protein-expressing cells were seeded at 1.5×10⁵ cells per well in 96 well V-bottom plates. The cells were first incubated with human serum (diluted 1:100 in 10% FBS) before a secondary incubation with a double stain, consisting of Alexa Fluor 647-conjugated secondary antibodies (diluted 1:500) and propidium iodide (PI; diluted 1:2500). Secondary antibodies used are conjugated anti-human IgM, or IgG. For assays examining IgG subclasses, the secondary incubation was with mouse anti-human IgG1, IgG2, IgG3, or anti-human IgG4. Following the secondary incubation, the cells were then incubated with Alexa Fluor 647-conjugated anti-mouse IgG. Cells were read on BD Biosciences LSR4 laser and analyzed using FlowJo (Tree Star).

Linear Epitope IgG ELISA

The ELISA was performed as previously described (Amrun et al., 2020). Briefly, Maxisorp flat-bottom 96-well plates were coated overnight at 4° C. with 1:2000 dilution of NeutrAvidin protein (1 mg/ml). Plates were blocked with 0.01% Polyvinyl Alcohol (PVA) solution in 0.1% PBST (blocking buffer) before addition of pooled or single biotinylated peptides (1:2000 dilution in 0.1% PBST), and plasma samples (1:1000 dilution in 0.1% PBST). Goat anti-human IgG-HRP diluted in blocking buffer was used for detection of peptide-bound antibodies. Tetramethylbenzidine substrate was then added to the plates and the reaction was stopped using 0.16 M sulphuric acid. Absorbance measurements were done using wavelength (450 nm) on an Infinite M200 plate reader (Tecan, firmware V_2.02_11/06). In all steps, plates were incubated at RT for 1 h on a rotating shaker and washed twice with 0.1% PBST in between steps.

GenScript cPass Neutralization Antibody Detection Kit

Serum samples were analysed by the GenScript cPass Neutralization Antibody Detection kit, according to the manufacturer's instructions. Briefly, the serum samples were first diluted 1:10 in provided sample dilution buffer and then mixed with HRP-conjugated RBD with a volume ratio of 1:1 and incubated at 37° C. for 30 min. The mixture was added to wells in the capture plate in the kit for another incubation at 37° C. for 15 min. After washing, TMB solution was added to the plate and the plate was incubated in the dark for 15 min at 25° C. Absorbance at 450 nm was read immediately with Sunrise Microplate Reader (Tecan) after the addition of the stop solution. Samples were defined as positive when the inhibition is ˜20%−inhibition was calculated as Inhibition=(1−OD value of Sample/OD value of Negative Control)×100%, according to manufacturer's instructions. Reading of 10-30% inhibition was defined as borderline results.

WondFo SARS-CoV-2 IgG/IgM Rapid Diagnostic Test (RDT)

Serum samples were analysed by the WondFo SARS-CoV-2 IgG/IgM RDT according to the manufacturer's instructions. Briefly, serum samples were first added to the sample wells before the addition of the detection buffer into the buffer well. The test kit was then left at room temperature for 15 min before being visually read.

Quantification and Statistical Analysis

Quantification of S Protein Antibody by Flow Cytometry

Binding of specific antibody binding to cells were determined by LSR4 laser (BD Biosciences) and analyzed using FlowJo (Tree Star). Cells were gated on: (1) FSC-A/SSC-A to exclude cell debris (FIG. 8A), (2) FSC-A/FSC-H to select for single cells (FIG. 8B), (3) FSC-A/PI to select for live cells (PI-negative population, FIG. 8C), (4) FITC/Alexa Fluor 647 (FIG. 8D-H). Binding is determined by the percentage of GFP-positive S protein-expressing cells that are bound by specific antibody, indicated by the events that are Alexa Fluor 647- and FITC-positive (Gate 2). A sample is defined as positive when the binding is more than mean+3SD of the healthy controls (n=22).

Using the cohort of 81 COVID-19 patients (Table 2), the sensitivity, or true positive rate, of the assay was defined by (true positive)/(true positive+false negative), expressed as a percentage. Specificity, or true negative rate, of the SFB assay was defined by (true negative)/(true negative+false positive), expressed as a percentage. For calculations on specificity, three control groups were included: recovered SARS (n=20), healthy controls (n=22) and seasonal human CoV (n=20).

TABLE 2
Demographic and clinical information of symptomatic
COVID-19 patients, healthy donors, recovered
SARS, seasonal human CoV patients, and samples
from National Public Health laboratory (NPHL)

| Symptomatic COVID-19 patients | Patients (N = 81) | |
|---|---|---|
| Age, Mean years (SD) | 45 | (13) |
| Gender, n (%) | | |
| Male | 48 | (59.3%) |
| Female | 33 | (40.7%) |
| Ethnicity, n (%) | | |
| Chinese | 68 | (84.0%) |
| Others | 13 | (16.0%) |
| Co-morbidities, n (%) | | |
| Diabetes | 7 | (8.6%) |
| Hypertension | 15 | (18.5%) |
| Others | 11 | (13.6%) |
| Vital signs at admission | | |
| Temperature, Mean ° C., (SD) | 37.7 | (0.9) |
| Heart rate, Mean beats/minute (SD) | 91.4 | (16.6) |
| Respiratory rate, Mean rate per minute (SD) | 18.4 | (1.9) |
| Diastolic blood pressure, Mean mmHg (SD) | 97.5 | (2.4) |
| Systolic blood pressure, Mean mmHg (SD) | 132.2 | (18.5) |
| Oxygen saturation, Mean % (SD) | 77.8 | (15.2) |
| Laboratory findings | | |
| Haemoglobin, Mean g/dL (SD) | 13.8 | (1.6) |
| Haematocrit, Mean % (SD) | 40.8 | (4.6) |
| Platelets, Mean × 10⁹/L (SD) | 194.8 | (69.8) |
| White blood cells, Mean × 10⁹/L (SD) | 5.3 | (3.0) |
| Lymphocytes, Mean × 10⁹/L (SD) | 1.2 | (0.6) |
| Neutrophils, Mean × 10⁹/L (SD) | 4.3 | (7.7) |
| Monocytes, Mean × 10⁹/L (SD) | 0.6 | (1.1) |
| C-reactive protein, Mean mg/L (SD) | 37.4 | (55.7) |
| Creatinine, Mean μmol/L (SD) | 75.0 | (45.3) |
| Lactate dehydrogenase, Mean U/L (SD) | 514.3 | (298.4) |
| Alanine aminotransferase, Mean U/L (SD) | 34.6 | (28.1) |
| Clinical outcome, clinical severity; group | | |
| No pneumonia, 0; mild, n (%) | 34 | (42.0) |
| Pneumonia without hypoxia, 1; moderate, n (%) | 28 | (34.5) |
| Pneumonia, with hypoxia, 2; severe, n (%) | 19 | (23.5) |
| Healthy donors | N = 22* | |
| Age, Mean years (SD) | 45 | (13) |
| Gender, n (%) | | |
| Male | 9 | (40.9%) |
| Female | 12 | (54.5%) |
| Recovered SARS | N = 20 | |
| Age, Mean years (SD) | 48 | (13) |
| Gender, n (%) | | |
| Male | 5 | (25.0%) |
| Female | 15 | (75.0%) |
| Seasonal human CoV | N = 20 | |
| Age, Mean years (SD) | 44 | (16) |
| Gender, n (%) | | |
| Male | 7 | (53.8%) |
| Female | 6 | (46.2%) |

| | |
|---|---|
| NPHL Samples | N = 108 |
| PCR-positive | |
| Symptomatic | 16 |
| Convalescent COVID-19 patients | 14/16 |
| Random testing of general population | 2/16 |
| No symptom | 34 |
| MOH** surveillance of dormitory residents | 33/34 |
| MOH surveillance of suspected and quarantined | 1/34 |
| Symptom-unknown | 11 |
| MOH surveillance of suspected and quarantined | 11/11 |
| PCR-negative | |
| Symptom-unknown | 13 |
| MOH surveillance of suspected and quarantined | 13/13 |
| PCR-negative/not done | |
| No Symptom | 20 |
| MOH surveillance of dormitory residents | 20/20 |
| PCR-unknown | |
| Symptom-unknown | 15 |
| MOH surveillance of suspected and quarantined | 9/15 |
| Random testing of general population | 6/15 |

*Information of one donor unknown.
**Ministry of Health.
SARS: Severe Acute Respiratory Syndrome Coronavirus;
human CoV: human coronavirus

Statistical Analysis

Statistical analysis was done using GraphPad Prism (GraphPad Software). For comparing between multiple groups, Kruskal-Wallis tests and post hoc tests using Dunn's multiple comparison tests were used to identify significant differences. For paired comparison between total IgG and IgG1 response, Wilcoxon matched-pairs signed rank test was used. P-values less than 0.05 are considered significant, where * indicates P≤0.05, ** indicates P≤0.01, *** indicates P≤0.001, **** indicates P≤0.0001.

TABLE 4
Key resource table

| | | |
|---|---|---|
| REAGENT or RESOURCE | SOURCE | IDENTIFIER |

Antibodies

| | | |
|---|---|---|
| Anti-human IgG Alexa Fluor 647 | Thermo Fisher | Cat# A21445 |
| | Scientific | |
| Anti-human IgM Alexa Fluor 647 | Thermo Fisher | Cat# A21249 |
| | Scientific | |
| Anti-mouse IgG Alexa Fluor 647 | Thermo Fisher | Cat# A21235 |
| | Scientific | |
| Anti-human IgG1 | Thermo Fisher | Cat# MA1-34581 |
| | Scientific | |
| Anti-human IgG2 | BioLegend | Cat# 411102 |
| Anti-human IgG3 | BioLegend | Cat# 411302 |
| Anti-human IgG4 | Thermo Fisher | Cat# A10651 |
| | Scientific | |
| Anti-human IgG-HRP | Jackson | Cat# 109-035-218 088 |
| | ImmunoResearch | |

Bacterial and Virus Strains

| | | |
|---|---|---|
| XL10 gold ultracompetent bacterial | Agilent | Cat# 200314 |
| cells | | |
| XL10 bacterial cells harbouring pHIV- | Generated in | NA |
| SARS-CoV-2-SP-eGPF | this study | |

Biological Samples

| | |
|---|---|
| Plasma samples from symptomatic | IRB# |
| COVID-19 patients | 2020/00091 |
| Plasma samples from recovered SARS | IRB# |
| patients | 2012/00917 |
| Plasma samples from seasonal human | IRB# |
| CoV patients | 2020/00076 |
| Plasma samples from healthy donors | IRB# |
| | 2017/2806 |
| | and IRB# 04-140 |
| | Singapore |
| | Infectious |
| | Diseases Act |

Chemicals, Peptides, and Recombinant Proteins

| | | |
|---|---|---|
| EndoFectin Lenti | GeneCopoeia | Cat# EF001 |
| Polybrene | Sigma-Aldrich | Cat# H9268 |
| Dulbecco's Modified Eagle Medium | HyClone | Cat# |
| (DMEM) | | SH30022.01 |
| Fetal Bovine Serum (FBS) | HyClone | Cat# |
| | | SV30160.03 |
| | | HI |
| Propidium Iodide | Sigma-Aldrich | Cat# P4170 |
| ACE2-human Fc | Prof Wang | NA |
| | Cheng-I's | |
| | laboratory | |
| NeutrAvidin protein | Thermo Fisher | Cat# 31050 |
| | Scientific | |
| 0.01% Polyvinyl Alcohol | Sigma-Aldrich | Cat# 341584 |
| S14P5 biotinylated peptides | Mimotopes | NA |
| S20P2 biotinylated peptides | Mimotopes | NA |
| S21P2 biotinylated peptides | Mimotopes | NA |
| N4P5 biotinylated peptides | Mimotopes | NA |
| Tetramethylbenzidine substrate | Sigma-220 | Cat# T8665 |
| | Aldrich | |
| Sulphuric acid | Merck | Cat#1.00731.1000 |

Critical Commercial Assays

| | |
|---|---|
| cPass Neutralization Antibody | GenScript |
| Detection kit | |
| SARS-CoV-2 IgG/IgM Rapid diagnostic | Guangzhou |
| test (RDT) | WondFo |
| | Biotech |

Experimental Models: Cell Lines

| | | |
|---|---|---|
| HEK293T | ATCC | Cat# CRL-3216 |
| HEK293T expressing full length S | Generated in | NA |
| protein | this study | |

Oligonucleotides

| | | |
|---|---|---|
| EF1aFor | Integrated | EF1aFor |
| | DNA | |
| | Technologies | |
| SPseqF1 | Integrated | SPseqF1 |
| | DNA | |
| | Technologies | |
| SPseqF2 | Integrated | SPseqF2 |
| | DNA | |
| | Technologies | |
| SPseqF3 | Integrated | SPseqF3 |
| | DNA | |
| | Technologies | |
| SPseqF4 | Integrated | SPseqF4 |
| | DNA | |
| | Technologies | |
| SPseqR1 | Integrated | SPseqR1 |
| | DNA | |
| | Technologies | |
| IRESrev | Integrated | IRESrev |
| | DNA | |
| | Technologies | |

Plasmids

| | | |
|---|---|---|
| pHIV-eGFP | Addgene | Cat# 21373 |
| pMD2.G | Addgene | Cat# 12259 |
| pMDLg/pRRE | Addgene | Cat# 12251 |
| pRSV-Rev | Addgene | Cat# 12253 |
| pHIV-SARS-CoV-2-SP-eGPF | Generated in | NA |
| | this study | |

Others

| | | |
|---|---|---|
| BD Vacutainer ® CPT ™ tubes | BD | Cat# 362753 |
| | Biosciences | |
| 96 V-bottomed well plates | Thermo Fisher | Cat# 249570 |
| | Scientific | |
| Maxisorp flat-bottom 96-well plates | Thermo Fisher | Cat# 442404 |
| | Scientific | |

Results

Profiles of Specific Antibodies Against Full Length S Protein Over the Course of

Infection

To characterize the antibody profile of COVID-19 patients, the inventors of the present disclosure developed a flow cytometry-based assay based on the full length SARS-CoV-2 S protein (SFB assay), which allows the detection of a wider repertoire of antibodies such as antibodies binding to various domains and conformational epitopes of the S protein. To this end, HEK293T cells was transduced with lentiviral particles to express the full length S protein on the cell surface. The expression of the S protein on the cell surface was verified by examining the binding of the ACE2-huFc (ACE2 protein tagged with a human Fc) and S protein RBD (receptor-binding domain)-specific monoclonal antibody clone 5A6 to the S protein-expressing cells (FIG. 7).

Using the S protein-expressing cells, the inventors examined the antibody response against the full length S protein in symptomatic COVID-19 patients (n=81; Table 2) over the course of infection, at time points with a median of 5 days, 10 days and 23 days post-infection onset (pio). Various groups of controls (Table 2) were also assessed in parallel: (1) recovered SARS individuals (n=20), (2) healthy controls (n=22), and (3) seasonal human CoV patients (n=20). Specific IgM against the S protein was detected COVID-19 patients, with the response being higher at time point with a median of 10 days pio than at time point with a median of 5 days pio (FIG. 1A). Specific IgM was not detected in the control groups. A total of 19% of the patients had a positive IgM response as early as median of 5 days pio (FIG. 1C). By median of 10 days pio, 61% of the patients acquired IgM. Notably, specific IgM was not detected in all control groups, highlighting the specificity of the assay (FIGS. 1A and 1C). Similar to IgM, the specific IgG response was higher at time point median 23 days pio than at median 10 days pio (FIGS. 1B and 1D). IgG seroconversion was detected in 86% of the patients at median 10 days pio (FIG. 1D) and 100% seroconversion was achieved by median 23 days pio. Four recovered SARS individuals demonstrated IgG cross-reactivity against the SARS-CoV-2 S protein (FIGS. 1B and 1D).

The present inventors have previously also reported the antibody response of the same cohort of patients using linear epitopes IgG ELISA (Amrun et al., 2020). Similarly, specific IgG (FIG. 8) were observed. Comparing the SFB assay and the S protein linear epitope IgG ELISA (S14P5, S20P2, S21P2), the SFB assay detected a greater proportion of patients at both time points (FIG. 8). Interestingly, the N protein linear epitope ELISA (N4P5) detected specific IgG in 98% of the patients as early as 10 days pio, suggesting that the kinetics in the production of specific antibody against the N protein might be different.

IgG1 was the Dominant IgG Subclass Specific Against S Protein

The inventors of the present disclosure went on to study the specific S protein IgG subclasses profile of COVID-19 patients. All four IgG subclasses responses were detected in the patients, with responses being higher at median 23 days pio than at median of 10 days pio (FIG. 2A-D). There was a dominance of IgG1 response, followed up by IgG3, IgG2 and lastly, IgG4 (FIGS. 2E and 2F). IgG subclasses seroconversion was lower at median 10 days (FIG. 2G). By median 23 days pio, all patients had a positive IgG1 response, while 74%, 94% and 67% of patients had a positive IgG2, IgG3 and IgG4 response (FIG. 2H). This demonstrated isotype switching of all IgG subclasses against the S protein over time in the COVID-19 patients, with IgG1 response being the most dominant IgG subclass.

SFB Assay is Specific and Sensitive

The inventors of the present disclosure next assessed its utility of the SFB assay for diagnosis of SARS-COV-2. Using the control groups (recovered SARS (n=20), healthy control (n=22) and seasonal human CoV (n=20)), the specificity of the SFB assay was 100% and 94% for IgM and IgG detection respectively (Table 1). There was no cross-reactivity observed with IgM detection. For IgG, cross-reactivity was observed in 4/20 recovered SARS patients, but not in healthy controls and seasonal human CoV patients. The specificity of the SFB assay was 97%, 98%, 98% and 98% for IgG1, IgG2, IgG3 and IgG4 respectively. For IgG1, the inventors observed cross-reactivity with 1/20 recovered SARS patients and 1/22 healthy controls. For IgG2, IgG3 and IgG4, cross-reactivity was detected in 1/22 healthy controls.

Using the cohort of 81 COVID-19 patients (Table 2), while the sensitivity of the SFB assay for IgM detection was lower (19% and 61% at time point with a median of 5 days pio and 10 days pio respectively), the SFB assay was more sensitive for IgG detection, 86% and 100% at time point with a median of 10 days pio and 23 days pio respectively (Table 1). Unsurprisingly, at time point median 10 days pio where antibody responses were lower, the sensitivity of the SFB assay for IgG1, IgG2, IgG3 and IgG4 detection was lower, at 64%, 37%, 46 and 32% respectively. At a later time point of median 23 days pio, the SFB assay was more sensitive, 100% and 94% for IgG1 and IgG3 detection, but less sensitive for IgG2 and IgG4 detection, 74% and 67% respectively.

TABLE 1
Receiver operating characteristic (ROC)
profiles of specific S protein antibodies

| | | Threshold* | Sensitivity | Specificity | |
|---|---|---|---|---|---|
| | Isotype | (%) | (%) | (%) | AUC |
| Median | IgM | 11.28 | 32.35 | 100.00 | 0.702 |
| 5 days | | | | | |
| pio | | | | | |
| Median | IgM | 11.28 | 62.71 | 100.00 | 0.881 |
| 10 days | IgG | 6.93 | 86.44 | 93.44 | 0.942 |
| pio | IgG1 | 15.57 | 66.10 | 96.72 | 0.900 |
| | IgG2 | 7.04 | 37.29 | 98.36 | 0.868 |
| | IgG3 | 3.50 | 45.76 | 98.36 | 0.885 |
| | IgG4 | 6.38 | 32.20 | 98.36 | 0.858 |
| Median | IgG | 6.93 | 100.00 | 93.44 | 1.000 |
| 23 days | IgG1 | 15.57 | 100.00 | 96.72 | 1.000 |
| pio | IgG2 | 7.04 | 74.24 | 98.36 | 0.988 |
| | IgG3 | 3.50 | 93.94 | 98.36 | 0.996 |
| | IgG4 | 6.38 | 62.71 | 98.36 | 0.976 |

*Threshold is defined as mean + 3SD of healthy controls (n = 22)

SFB Assay can Detect Pre-/Asymptomatic Infections

Having established the SFB assay for serological analysis of symptomatic infections, the inventors proceeded to further examine the sensitivity of the assay and evaluate the feasibility of the assay for serological diagnosis of samples received at a diagnostic laboratory where there might be limited information on the samples. The Singapore National Public Health Laboratory (NPHL) received samples collected from convalescent cases, suspected infections and general populations for sero-prevalence studies. A total of 109 samples, grouped by PCR-status and symptom status (Table 2), were screened: (1) PCR-positive and symptomatic, n=16, (2) PCR-positive and pre-/asymptomatic, n=34, (3) PCR-positive and unknown symptom status, n=11, (4) PCR-negative and unknown symptom status, n=13, (5) PCR status-negative/not done and no symptom, n=20, (6) PCR status-unknown and unknown symptom status, n=15.

In agreement with the findings on the earlier cohort of 81 COVID-19 patients, the SFB assay detected IgM (FIG. 3A) and IgG (FIG. 3B), and also IgG subclasses (FIG. 3C-F) against the S protein in the PCR-positive and symptomatic infections. More importantly, the assay also detected specific IgM and IgG, and IgG subclasses against the S protein in PCR-positive and pre-/asymptomatic infections, with IgG1 being the dominant IgG subclass. The antibody response in this group was significantly lower than that observed with samples from PCR-positive and symptomatic infections, for all isotypes. Similarly, for the rest of the four groups, all isotypes responses were also lower than that observed with samples from PCR-positive and symptomatic infections. IgG1 was also the dominant IgG subclass observed, while the levels of IgG2 and IgG4 were negligible. Interestingly, for all groups, a greater IgG1 response was observed as compared to total IgG response (FIG. 4). This suggests that the IgG1 detection with SFB assay might provide greater sensitivity.

Comparison of the SFB with Commercially Available Serological Assays

All samples received at NPHL were first assessed by two commercially available serological assays: (1) GenScript cPass S protein RBD Neutralization Antibody Detection kit due to its ability to detect neutralising antibodies targeting the RBD of the S protein, and (2) Wondfo SARS-CoV-2 IgG/IgM rapid diagnostic test (RDT) with undisclosed antigen specificity, due to the rapid test format and ease of application to high throughput screening. With the exception of the samples collected from PCR-positive and symptomatic infections (which were tested positive with the two commercial antibody assays), most of the remaining 92 samples yielded either borderline or discrepant results with the two commercial assays, and were selected for analysis by the SFB assay.

In comparison, the SFB assay was found to be highly sensitive. With PCR-positive and symptomatic infections, the all isotypes SFB assay was comparable to the two commercially available assays, detecting 100% of the infections (FIG. 3G). More importantly, the all isotypes SFB assay was able to detect 97% of PCR-positive and pre-/asymptomatic infections, as compared to 32% and 35% with the cPass and RDT assays respectively. For PCR-positive and unknown symptom status samples, the all isotypes SFB assay detected 100% of samples, as compared with 18% with the cPass assay and 100% with the RDT assay. Similarly, for the rest of the three groups, the SFB assay was more sensitive than the cPass and RDT assays (FIG. 3G), being able to detect 62% of the PCR-negative and unknown symptom status samples, 95% of the PCR-negative/not done and no symptom infections, and 80% of the PCR-unknown and unknown symptom status samples. The finding demonstrated the high sensitivity of the SFB assay, allowing the assay to serve as an instrumental tool to detect asymptomatic infections.

Antibodies Against S Protein Associated with Disease Severity

Using other serological approaches such as ELISA, recent studies have reported association between high levels of specific antibodies against S protein and N protein and disease severity (Amrun et al., 2020; Long et al., 2020a; Okba et al., 2020; Qu et al., 2020; Zhang et al., 2020). Thus it was also investigated if this association between specific IgM and IgG against the full length S protein and disease severity was also present in the current cohort and if this association was specific of particular IgG isotypes. This is particularly relevant since Type 2 response (exemplified by high level of IgG2 and IgG4) has been hypothesised to be linked with enhanced disease (Arvin et al., 2020; de Alwis et al., 2020; Liu et al., 2019).

The patients were classified into three groups: mild (no pneumonia, clinical severity 0), moderate (pneumonia with no hypoxia, clinical severity 1), or severe (pneumonia with hypoxia, clinical severity 2) (Wong et al., 2020). Unsurprisingly, at median 5 days pio, where the IgM responses were low, any significant difference between the three disease severity groups was not observed (FIG. 5A). By median 10 days pio, specific IgM responses were associated with disease severity, where patients in the severe group showed significantly higher IgM responses (FIG. 5B). Specific IgG responses were also associated with disease severity at both time points with a median 10 and 23 days pio (FIGS. 5C and 5D). It was further examined if the specific IgM and IgG responses were associated with specific clinical outcomes such as pneumonia (FIGS. 9A and 9D), oxygen supply requirement (FIGS. 9B and 9E) and intensive care unit (ICU) admission (FIGS. 9C and 9F). While association was only found between specific IgG response and pneumonia at time point with a median of 23 days pio, the association of both specific IgM and IgG responses with more severe clinical outcomes such as oxygen supply requirement and ICU admission was observed as early as time point with a median of 10 days pio.

It was also observed an association between all specific IgG subclasses and the severity of the disease at both time points of median 10 days and 23 days pio (FIG. 6) with no specific isotype imbalance. Higher IgG1, IgG2, IgG3 and IgG4 responses were detected in patients with pneumonia and patients requiring ICU admission at time point with a median of 23 days pio, but not at median 10 days pio (FIG. 10A, 10D, 10G, 10J). In contrast, the association between higher responses for all IgG subclasses and oxygen supply requirement was found as early as 10 days pio (FIG. 10B, 10E, 10H, 10K).

The current strategy for controlling the COVID-19 pandemic requires confining world's population, which is not sustainable in the long term. The gradual easing of control measures will require active surveillance of the population to ensure early detection of new infections, contact tracing and quarantine and continued social distancing measures to block transmission. Serological assays are instrumental in confirming symptomatic infections and detecting individuals who are pre-symptomatic, asymptomatic or have recovered.

The inventors have previously developed an ELISA-based serological assay on the four immunodominant IgG linear epitopes on the S and N protein (Amrun et al., 2020). In order to capture a wider repertoire of antibodies against the S protein, a flow cytometry assay was developed based on the full length S protein in this study. The assay is more time-efficient—results are available within two hours. The assay is also well suited to detect anti-S protein antibodies in symptomatic patients. Symptomatic COVID-19 patients acquired specific IgM and IgG over the course of infection, with all patients having a detectable antibody response at a later stage of infection (median of 23 days pio) (FIG. 1).

One defining feature of the assay is its ability to detect specific IgG subclasses against the S protein. In the cohort of the present disclosure, it was found that all IgG subclasses were acquired by COVID-19 patients, with IgG1 was the most dominant IgG subclass. Similar to total IgG, the IgG subclasses response was significantly greater at the later stage of infection and was also strongly associated with disease severity. This is in agreement with a study from Ni et al (Ni et al., 2020), who also found a predominant IgG1 response against the RBD of the S protein and also the N protein. The inclusion of IgG subclasses in serological detection is important and bridges knowledge gaps in understanding the protective immunity against COVID-19 and the likelihood of protection from a re-infection. IgG1 and IgG3 induction, typically indicative of a TH1 response (Kawasaki et al., 2004), is a pro-inflammatory response particularly important in protective immunity against viruses. IgG1 and IgG3 possess higher neutralisation capabilities against many different viruses (Hofmeister et al., 2011; Richardson et al., 2019; Walker et al., 2020).

With increasing reports of asymptomatic individuals having similar transmission capability as symptomatic individuals (Bai et al., 2020; Hu et al., 2020), it is clear that asymptomatic infections play a role in transmission and it is crucial to stem asymptomatic transmission as well for effective COVID-19 disease control. The inventors of the present disclosure found that the SFB assay was able to detect PCR-positive and pre-/asymptomatic infections efficiently. The SFB assay could be a more effective tool for detecting COVID-19 infections and might be more informative at determining exposure. While the sensitivity for the cPass assay (GenScript, 2020) and the RDT assay (Bilcare, 2020) was reported to be 94% and 86% respectively, the SFB assay was found to be more sensitive and was able to detect specific antibodies in cases where discrepant or borderline results were achieved on the commercial cPass and RDT assay. Specific IgM and IgG were detected, with IgG1 being the most dominant IgG subclass. The antibody response was significantly lower in the PCR-positive and pre-/asymptomatic patients, which is in agreement with a recent study (Long et al., 2020b). Despite the lower antibody levels, the assay was able to detect 97% of these infections, where the cPass and RDT did not yield clear serological outcomes. This showed that more sensitive assays such as SFB assay are needed to detect pre-/asymptomatic infections, where the antibody response is weaker, and especially for determining exposure in the population, The higher sensitivity of the SFB assay over the cPass assay could be attributed to the target of the assay—the SFB assay is based on full length S protein, which allows capture of antibodies against various domains and also conformational epitopes, while the cPass assay was designed to detect specifically neutralising antibodies against the RBD of the S protein that block the interaction between the RBD domain and ACE2, its receptor on the host cell. The target of the RDT has not been disclosed. It is possible that it is also based on a particular domain of the S protein, hence capturing a smaller repertoire of S protein antibodies. It is also possible that the target is a different protein such as the N protein, and thus recognising a different set of antibodies with different kinetics in antibody induction. Of importance, IgG1 subclass detection by the SFB assay was key to ascertain exposure to the virus and provide better sensitivity than testing IgG alone—a significantly greater IgG1 response, as compared to total IgG, was observed in all groups (FIG. 4). It could be due to the secondary antibodies as the secondary anti-human IgG antibodies might not bind to all IgG subclasses with similar efficiency. More importantly, the greater IgG1 response (over total IgG) could be due to a prozone effect, where high levels of antibodies results in lack of antigen binding (Goh et al., 2015; Lieberman et al., 1988; Taborda et al., 2003; Zollinger and Mandrell, 1983). There have been studies reporting rapid decay of specific IgG against SARS-CoV-2 as early as one month pio (Ibarrondo et al., 2020; Seow et al., 2020). While there is likely a decrease in IgG levels over time, it is also possible that the lack of IgG binding could be, in part, due to prozone effect. Hence, the detection of a subset of total IgG, the IgG1 antibodies, might be better than the whole repertoire of total IgG.

Serological assays are complementary to PCR assays for COVID-19 diagnosis. As a preliminary evaluation of the SFB assay for ongoing sero-epidemiological studies, the present disclosure tested potentially exposed individuals who were PCR-negative from the NPHL cohort. A total of 13 such samples were borderline positive or discrepant with the cPass and RDT assays. The SFB assay detected 8/13 (62%) of these cases indicating that a fraction of them have been infected/exposed to the virus. Most of Singapore's COVID-19 cases have been among the migrant worker population (Chew et al., 2020) living in dormitories where social distancing is difficult. As part of Singapore Ministry of Health (MOH) surveillance on the dormitory residents, samples were collected from dormitory residents with no symptoms and a PCR-negative/not done status to assess transmission in dormitories. A total of 20 samples were borderline or discrepant by the cPass and RDT. The SFB assay detected 95% of these cases. These findings showed that serological assays, used in combination, are critical diagnostic tools to complement PCR assays and also further demonstrated the high sensitivity of the SFB assay.

While high sensitivity is needed to detect asymptomatic infections, it is also important to have high specificity. One main limitation of serological assays is the risk of false positive diagnosis. As the SFB assay consists of six tests (IgM, IgG, and four IgG subclasses), it allows internal validation. For 98/109 samples tested, a positive response was detected for two or more isotypes. 11/109 samples tested had a positive response for only one isotype, where 4/11 were also found to be positive by the cPass or RDT test and another 4/11, though negative by the cPass and RDT test, were PCR-positive. This showed that borderline positive results should be interpreted with caution. One other limitation of the SFB assay is that it is a cell-based assay. The dependence on cell culture requires planning ahead to ensure sufficient cell count, limiting the application of the assay for high throughput screening. Serological assays complement each other to provide better diagnosis—the cPass and RDT assays, which allows HTS, could serve as the first round of screening, and the more sensitive SFB assay could provide confirmation and further investigation of borderline/discrepant samples. The methods as described herein can be adapted to be an assay to detect all six isotypes in one single test using different fluorophores. This advantageously reduces the number of tests per sample. It is also noteworthy, while a large-sized flow cytometer (LSR4, BD Biosciences) was used in this study, the assay can also be developed for portable flow cytometers such as the Accuri (BD Biosciences), which could be deployed in small laboratory settings in places such airport or borders.

The inventors of the present disclosure have also observed that the antibody response was also strongly associated with disease severity. This is also in agreement with other recent studies, showing an association between antibody levels and disease severity (Amrun et al., 2020; Long et al., 2020a; Okba et al., 2020; Qu et al., 2020; Zhang et al., 2020). The antibody response was not associated with an isotype imbalance of IgG2 or IgG4 over IgG1 or IgG3 antibodies. IgG2 and IgG4 induced by a type II cytokine response been hypothesised to be able to mediate either viral infection enhancement or disease enhancement (Arvin et al., 2020; de Alwis et al., 2020; Liu et al., 2019). However, it is found that asymptomatic patients had barely any IgG2 and IgG4. Thus it is possible that despite high levels of IgG1 and IgG3, the level of IgG2 and IgG4 against the S protein observed in severe patients may be in part responsible for the as recently proposed in a pre-print study (Hoepel et al., 2020). This is an extremely important issue which deserves further studies.

Taken together, the current findings demonstrated that the SFB could be used for serological confirmation of symptomatic infections. The SFB could also be used, in combination with other serological assays, to detect asymptomatic infections and access sero-prevalence in the community. This would be an instrumental tool for sero-surveillance and provide crucial insight to the extent of undetected and undiagnosed COVID-19 cases in the community. In addition, the SFB assay could also be used for examination of the antibody response in previously infected individuals long after they have recovered to have a better understanding of the persistence of antibody-mediation protection against COVID-19. The high sensitivity of the SFB assay is also particularly useful in clinical investigation of suspected infections and epidemiological link within clusters, which might yield borderline/discrepant results or even be missed by less sensitive serological assays. It aids in contract tracing efforts to limit the extent of community spread. This is especially pertinent at a time when governments around the world are looking to gradually reopen the economy. This would greatly help to form better public policy decisions to manage and limit COVID-19 infections.

### Example 2

### Materials:

1.5 ml microcentrifuge tubes
1×PBS buffer
Goat anti-human IgG Alexa Fluor 647 (Thermofisher)
Goat anti-human IgM Alexa Fluor 647 (Thermofisher)
TritonX-inactivated plasma from healthy controls, acute and recovered COVID patients

### Storage:

Stained samples will be stored at 4° C. in the dark until acquisition.

### Preparation & Transportation:

Human samples have to be stored in triple biohazard ziplock bags (on ice) with secondary containment (cooler box) for transportation.

### General Guidelines:

1. Surfaces of the Biosafety Cabinet (BSC) and laboratory equipment (i.e. pipette guns, micropipettes) are to be wiped down with 1% Virkon solution before and after use.
2. Personnel are to don the respective PPE as follows:
   a. Disposable lab coat, safety goggles, face mask, hair net cover, shoe covers, double gloves (latex inside, nitrile outside).
3. Place biohazard bag (double bagged), waste bottle with 1% Virkon solution, and a container containing a multi-fold towel and 1% Virkon solution in BSC for wipe down work.
4. Segregate BSC working area into (1) clean, (2) working, and (3) waste zones.
5. Decontaminate used serological pipettes by aspirating 1% Virkon and leaving it in the waste container before disposal into the double-bagged biohazard waste bin (placed in BSC).
6. Anything that needs to be taken out of the BSC should be decontaminated via 1% Virkon solution in these steps:
   a. Wet a multi-fold towel with 1% Virkon solution and wipe gloves.
   b. Decontaminate an area within the clean zone and wipe down surfaces of objects before placing in the decontaminated area.
   c. Wait for 5 minutes before taking them out of the BSC.
7. After completion, carry out disinfection by wiping down the walls and surfaces of the BSC, racks, pipettes and other equipment/objects with 1% Virkon solution (wait for 5 minutes) and 70% ethanol before removing them out of the BSC. Allow liquid waste in 1% Virkon to decontaminate overnight. Discard biohazard bag into a bigger biohazard bag and send for autoclaving.

### Procedures:

Staining of Cells with Plasma Samples for FACS (Done in BSC-II):
1. Wash EL4 cells expressing SARS-Cov-2 spike proteins with PBS
2. Dilute plasma samples 1:100. Gently flick tube to mix
   a. Note: all plasma samples are first triton-inactivated by LN team before using for this SOP
3. Add diluted plasma to EL4 cells, resuspend gently and incubate 30 mins at 4° C.
4. Wash 2× with PBS (centrifuge at 1500×rpm for 5 minutes).
5. Add diluted secondary antibody 1:500 (Goat anti-human IgG Alexa Fluor 647 or Goat anti-human IgM Alexa Fluor 647), resuspend gently and incubate 30 mins in the dark at 4° C.
6. Wash 3× with PBS (centrifuge at 1500×rpm for 5 minutes)

7. Remove supernatant, being careful not to disturb the cell pellet. Leave some volume behind to prevent the cells from drying out.
8. Store cells at 4° C. in the dark until acquisition on LSRII (4 lasers).

### Operation of the BD LSR II (4 Laser) Flow Cytometer:

1. Put the flow cytometer on standby.
2. Disconnect the waste, air line and sheath tubings from respective containers.
3. Discard waste and top up with 1 litre of bleach before reconnecting waste tubing.
4. Release pressure in sheath container and refill with PBS before reconnecting tubings.
5. Release clamp to remove any air bubbles in tubings.
6. Check the laser output before opening BD FACSDIVA software.
7. Run Rainbow Calibration ‘8 Peaks’ particles to check the emission fluorophores.
8. Pulse vortex each sample tube before acquiring, ensuring that threshold rate does not exceed 15,000 events/second.
9. Discard tubes into the biohazard waste container.
10. Repeat steps 8 and 9 for other samples and/or panels.
   a. Place a beaker filled with Virkon under the Sample Injection Port (SIP) in between samples to collect any residual backflow.
11. Re-run Rainbow Calibration ‘8 Peaks’ particles to check the emission fluorophores.
12. After acquisition, wash the machine in the following steps:
   a. Run FACS Clean Solution for 5 minutes, with flow rate set to ‘HIGH’;
   b. Run FACS Rinse Solution for 5 minutes, with flow rate to ‘HIGH’;
   c. Run water for 5 minutes, with flow rate set to ‘HIGH’.
13. Put cytometer to standby before shutting down both cytometer and computer.

### Waste Management:

Excess human samples will be discarded into 1% Virkon & left to decontaminate for at least 24 hours before disposal down the sink.
Disposable consumables (e.g. serological pipettes, tips, tubes & plates) are to be soaked in 1% Virkon prior to disposal in double-bagged biohazard waste bags.
All equipment to be decontaminated with 1% Virkon and 70% ethanol after use.
Biological Safety Cabinet to be decontaminated with 1% Virkon after use and UV-ed before and after every experiment.

### Example 3

HEK293T cells were transduced with lentiviral particles to stably express the S protein of SARS-CoV2 on the cell surface. S protein-expressing cells were incubated with the patients' plasma for 30 minutes before incubating with anti-human antibodies conjugated with fluorophores for 20 minutes. Binding of specific antibodies was detected in flow cytometry (FIG. 1).

The inventors screened a cohort, consisting of 61 healthy controls and 66 patients (FIG. 1). There was no cross-reactivity with the healthy controls or seasonal CoV. While there is some cross-reactivity observed in SARS-CoV-1 recall patients (4/20), the levels were comparatively low. Specific IgG was detected in all SARS-CoV-2, where the specific IgG response of all 66 SARS-CoV-2 patients were significantly higher than the controls.

The established assay can also be applied to detect other isotypes. The inventors validated the assay for IgM detection (FIG. 1), which could be important in detecting patients in early stages of infection or asymptomatic patients, respectively. While the IgM assay has lower sensitivity, where specific IgM was detected in 36/59 patients, there was no cross-reactivity observed with all controls including the SARS-CoV-1 patients. The IgM assay could compliment the IgG assay—the high specificity of IgM assay would imply any IgM positive test would indicate presence of infection.

The inventors have also validated the assay for the detection of IgG subclasses (FIG. 2). It has been well established that IgG1 and IgG3 are important in neutralising viruses. With the IgG subclasses assay, the inventors were able to detect the IgG subclasses in the patients' plasma. All patients tested (n=66) have a strong IgG1 response. 62/66 patients have an IgG3 response; 49/66 patients have an IgG2 response; 44/66 patients have an IgG4 response. The strong presence of neutralising IgG1 and IgG3 in these 66 patients, whom eventually recovered, suggests IgG1 and IgG3 are important for protection. Indeed, the inventors found that the IgG1 and IgG3 response strongly correlated with the neutralisation capacity of the antibodies in plasma (FIG. 11). In addition, it has been well-established that IgG1 and IgG3 presence is indicative of a TH1 response. TH1 response is a pro-inflammatory response and is particularly important in protective immunity against viruses. IgG2 and IgG4 responses, indicative of a TH2 response, is less effective in mediating protective immunity against viruses. With this assay, the application to detect the predominant IgG isotypes presence allows an understanding of the type of immune response the particular patient might have (TH1 or TH2). This would be important in identifying individuals who are immune and potentially protected. Knowing the IgG isotypes would be crucial in refining the current vaccine candidates against COVID-19.

The S flow assay is well suited to detect anti-S protein antibodies in symptomatic patients. In addition, the S flow assay could also be used to detect infections where specific antibodies are low and might not be detected in other serological tests. Using a cohort of samples, consisting of 20 borderline samples, the inventors found that the sensitivity of the S flow IgG/IgM assay was comparable to other serological test, being able to detect 70% (14/20) of the samples (Table 5). The S flow IgG1 assay was slightly more sensitive, being able to detect 75% (15/20) of the samples. Together, the combined S flow assay (IgG/M, IgG1, IgG2, IgG3 and IgG4) was able to detect 90% (18/20) of the samples. This demonstrated that the S flow assay could serve an additional avenue, either by itself or in combination with other serological assays, to detect borderline infections, which may go or may have gone undetected. This would provide crucial insight to the extent of undetected and undiagnosed COVID-19 cases in the community.

TABLE 5
Serological analysis of borderline samples by various serological tests
including the S flow assay.

| | Test1, IgG/M | Test2, IgG/M | Test3, IgG/M | S Flow, IgG/M | S Flow, IgG1 | S Flow, All |
|---|---|---|---|---|---|---|
| Positive, n = 29 | 29/29 (100%) | 29/29 (100%) | 29/29 (100%) | 29/29 (100%) | 29/29 (100%) | 29/29 (100%) |
| Negative n = 17 | 0/17 (0%) | 0/17 (0%) | 0/17 (0%) | 0/17 (0%) | 0/17 (0%) | 0/17 (0%) |
| Discrepant/borderline positive, n = 20 | 3/20 (15%) | 14/20 (70%) | 4/15 (27%) | 14/20(70%) | 15/20(75%) | 18/20(90%) |

only 15/20 tested by test3

### Example 4

Assay is based on the discovery that 1 out of 4 sub types of antibody IgG (IgG1) is dominant in COVID-19 patients, especially at median 23 days post infection.

Presence of IgG1 can clearly determine if a patient has a COVID-19 infection and is highly differentiating between COVID-19 patients and seasonal nCoV, SARS and healthy patients.

Method: SARS-CoV-2 gene is introduced to human cells together with the green fluorescent protein (GFP). Cells which produce the GFP and the viral S protein are separated using a high speed cell sorter (such as FACS ARIA). These cells are added to the COVID-19 patient serum. The S protein produced binds to the antibodies present in the serum and the bound antibodies are detected using a fluorescence reader.

FIG. 7 shows intensity of fluorescence collates with percentage antibodies present and FIG. 2 shows strong IgG1 presence over the other subtypes at day 10 and day 23 post infection.

### Example 5

Using the same approach described above, the inventors have further developed the assay by generating cell lines to express the S protein from new emerging variants of SARS-CoV-2. The new additions include the alpha and beta variants of the SARS-CoV-2.

The cells expressing the alpha and beta variants has been validated using a set of convalescent plasma samples from COVID-19 patients, where the inventors found that the antibody responses to both alpha and beta variants are significantly diminished, compared with the response to the wildtype (WT) (FIG. 12). The methods as disclosed herein may be used to examine potential vaccine efficacy against the alpha and beta variants by studying the antibody response against the variants in plasma samples collected from individuals following vaccination with COVID-19 vaccines.

In addition, the inventors have also developed the assay for the delta variant of SARS-CoV-2. The inventors found that, following vaccination with COVID-19 vaccines, vaccines developed a strong antibody response against the wildtype spike protein. However, the antibody response against the delta variant was significantly reduced, suggesting lower potential vaccine efficacy against delta variant (FIG. 13).

### REFERENCES

Ahmed, S. F., Quadeer, A. A., and McKay, M. R. (2020). Preliminary Identification of Potential Vaccine Targets for the COVID-19 Coronavirus (SARS-CoV-2) Based on SARS-CoV Immunological Studies. Viruses 12.
Amanat, F., Stadlbauer, D., Strohmeier, S., Nguyen, T. H. O., Chromikova, V., McMahon, M., Jiang, K., Asthagiri Arunkumar, G., Jurczyszak, D., Polanco, J., et al. (2020). A serological assay to detect SARS-CoV-2 seroconversion in humans. medRxiv.
Amrun, S. N., Lee, C. Y., Lee, B., Fong, S. W., Young, B. E., Chee, R. S., Yeo, N. K., Torres-Ruesta, A., Carissimo, G., Poh, C. M., et al. (2020). Linear B-cell epitopes in the spike and nucleocapsid proteins as markers of SARS-CoV-2 exposure and disease severity. EBioMedicine 58, 102911.
Arvin, A. M., Fink, K., Schmid, M. A., Cathcart, A., Spreafico, R., Havenar-Daughton, C., Lanzavecchia, A., Corti, D., and Virgin, H. W. (2020). A perspective on potential antibody-dependent enhancement of SARS-CoV-2. Nature 584, 353-363.
Bai, Y., Yao, L., Wei, T., Tian, F., Jin, D. Y., Chen, L., and Wang, M. (2020). Presumed Asymptomatic Carrier Transmission of COVID-19. Jama.
Bilcare (2020). Wondfo SARS-CoV-2 Antibody Test (https://www.bilcare.com/Wondfo/wondfo.html: Aug. 31, 2020).
Chew, M. H., Koh, F. H., Wu, J. T., Ngaserin, S., Ng, A., Ong, B. C., and Lee, V. J. (2020). Clinical assessment of COVID-19 outbreak among migrant workers residing in a large dormitory in Singapore. J Hosp Infect.
Chu, D. K. W., Pan, Y., Cheng, S. M. S., Hui, K. P. Y., Krishnan, P., Liu, Y., Ng, D. Y. M., Wan, C. K. C., Yang, P., Wang, Q., et al. (2020). Molecular Diagnosis of a Novel Coronavirus (2019-nCoV) Causing an Outbreak of Pneumonia. Clin Chem 66, 549-555.
Cohen, J., and Normile, D. (2020). New SARS-like virus in China triggers alarm. Science 367, 234-235.
Corman, V. M., Landt, O., Kaiser, M., Molenkamp, R., Meijer, A., Chu, D. K., Bleicker, T., Brunink, S., Schneider, J., Schmidt, M. L., et al. (2020). Detection of 2019 novel coronavirus (2019-nCoV) by real-time RT-PCR. Euro Surveil! 25.
de Alwis, R., Chen, S., Gan, E. S., and Ooi, E. E. (2020). Impact of immune enhancement on Covid-19 polyclonal hyperimmune globulin therapy and vaccine development. EBioMedicine 55, 102768.
GenScript (2020). cPass Neutralization Antibody Detection kit (https://www.genscript.com/covid-19-detection-svnt.html: Aug. 31, 2020).
GeurtsvanKessel, C. H., Okba, N. M. A., Igloi, Z., Bogers, S., Embregts, C. W. E., Laksono, B. M., Leijten, L., Rokx, C., Rijnders, B., Rahamat-Langendoen, J., et al. (2020). An evaluation of COVID-19 serological assays informs future diagnostics and exposure assessment. Nature communications 11, 3436.
Goh, Y. S., Clare, S., Micoli, F., Saul, A., Mastroeni, P., and MacLennan, C. A. (2015). Monoclonal Antibodies of a Diverse Isotype Induced by an O-Antigen Glycoconjugate Vaccine Mediate In Vitro and In Vivo Killing of African Invasive Nontyphoidal Salmonella. Infection and immunity 83, 3722-3731.
Hahn, S. M. (2020). Coronavirus (COVID-19) Update: FDA Expedites Review of Diagnostic Tests to Combat COVID-19. (2020). (https://www.fda.gov/news-events/press-an nouncements/coronavirus-covid-19-update-fda-expedites-review-diagnostic-tests-combat-covid-19: Aug. 1, 2020).
Hoepel, W., Chen, H.-J., Allahverdiyeva, S., Manz, X., Aman, J., Bonta, P., Brouwer, P., de Taeye, S., Caniels, T., van der Straten, K., et al. (2020). Anti-SARS-CoV-2 IgG from severely ill COVID-19 patients promotes macrophage hyper-inflammatory responses. bioRxiv, 2020.2007.2013.190140.
Hoffmann, M., Kleine-Weber, H., Schroeder, S., Kruger, N., Herrler, T., Erichsen, S., Schiergens, T. S., Herrler, G., Wu, N. H., Nitsche, A., et al. (2020). SARS-CoV-2 Cell Entry Depends on ACE2 and TMPRSS2 and Is Blocked by a Clinically Proven Protease Inhibitor. Cell 181, 271-280 e278.
Hofmeister, Y., Planitzer, C. B., Farcet, M. R., Teschner, W., Butterweck, H. A., Weber, A., Holzer, G. W., and Kreil, T. R. (2011). Human IgG subclasses: in vitro neutralization of and in vivo protection against West Nile virus. Journal of virology 85, 1896-1899.
Hu, Z., Song, C., Xu, C., Jin, G., Chen, Y., Xu, X., Ma, H., Chen, W., Lin, Y., Zheng, Y., et al. (2020). Clinical characteristics of 24 asymptomatic infections with COVID-19 screened among close contacts in Nanjing, China. Sci China Life Sci 63, 706-711.
Ibarrondo, F. J., Fulcher, J. A., Goodman-Meza, D., Elliott, J., Hofmann, C., Hausner, M. A., Ferbas, K. G., Tobin, N. H., Aldrovandi, G. M., and Yang, O. O. (2020). Rapid Decay of Anti-SARS-CoV-2 Antibodies in Persons with Mild Covid-19. The New England journal of medicine 383, 1085-1087.
Jiang, L., Lee, V. J., Cui, L., Lin, R., Tan, C. L., Tan, L. W., Lim, W. Y., Leo, Y. S., Low, L., Hibberd, M., et al. (2017). Detection of viral respiratory pathogens in mild and severe acute respiratory infections in Singapore. Scientific reports 7, 42963.
Kawasaki, Y., Suzuki, J., Sakai, N., Isome, M., Nozawa, R., Tanji, M., and Suzuki, H. (2004). Evaluation of T helper-1/-2 balance on the basis of IgG subclasses and serum cytokines in children with glomerulonephritis. Am J Kidney Dis 44, 42-49.
Lee, C. Y., Lin, R. T. P., Renia, L., and Ng, L. F. P. (2020). Serological Approaches for COVID-19: Epidemiologic Perspective on Surveillance and Control. Frontiers in immunology 11, 879.
Leong, H. N., Earnest, A., Lim, H. H., Chin, C. F., Tan, C., Puhaindran, M. E., Tan, A., Chen, M. I., and Leo, Y. S. (2006). SARS in Singapore—predictors of disease severity. Annals of the Academy of Medicine, Singapore 35, 326-331.
Lieberman, M. M., Frank, W. J., and Brady, A. V. (1988). Protective mechanism of the immune response to a ribosomal vaccine from Pseudomonas aeruginosa. II. In vitro bactericidal and opsonophagocytic studies with specific antiserum. The Journal of surgical research 44, 251-258.
Liu, L., Wei, Q., Lin, Q., Fang, J., Wang, H., Kwok, H., Tang, H., Nishiura, K., Peng, J., Tan, Z., et al. (2019). Anti-spike IgG causes severe acute lung injury by skewing macrophage responses during acute SARS-CoV infection. JCI insight 4.
Long, Q. X., Liu, B. Z., Deng, H. J., Wu, G. C., Deng, K., Chen, Y. K., Liao, P., Qiu, J. F., Lin, Y., Cai, X. F., et al. (2020a). Antibody responses to SARS-CoV-2 in patients with COVID-19. Nature medicine 26, 845-848.
Long, Q. X., Tang, X. J., Shi, Q. L., Li, Q., Deng, H. J., Yuan, J., Hu, J. L., Xu, W., Zhang, Y., Lv, F. J., et al. (2020b). Clinical and immunological assessment of asymptomatic SARS-CoV-2 infections. Nature medicine 26, 1200-1204.
Lv, H., Wu, N. C., Tsang, 0.T., Yuan, M., Perera, R., Leung, W. S., So, R. T. Y., Chan, J. M. C., Yip, G. K., Chik, T. S. H., et al. (2020). Cross-reactive antibody response between SARS-CoV-2 and SARS-CoV infections. bioRxiv.
Ni, L., Ye, F., Cheng, M. L., Feng, Y., Deng, Y. Q., Zhao, H., Wei, P., Ge, J., Gou, M., Li, X., et al. (2020). Detection of SARS-CoV-2-Specific Humoral and Cellular Immunity in COVID-19 Convalescent Individuals. Immunity 52, 971-977 e973.
Okba, N. M. A., Muller, M. A., Li, W., Wang, C., GeurtsvanKessel, C. H., Corman, V. M., Lamers, M. M., Sikkema, R. S., de Bruin, E., Chandler, F. D., et al. (2020). Severe Acute Respiratory Syndrome Coronavirus 2-Specific Antibody Responses in Coronavirus Disease Patients. Emerging infectious diseases 26, 1478-1488.
Pan, Y., Zhang, D., Yang, P., Poon, L. L. M., and Wang, Q. (2020). Viral load of SARS-CoV-2 in clinical samples. The Lancet Infectious diseases 20, 411-412.
Pung, R., Chiew, C. J., Young, B. E., Chin, S., Chen, M. I., Clapham, H. E., Cook, A. R., Maurer-Stroh, S., Toh, M., Poh, C., et al. (2020). Investigation of three clusters of COVID-19 in Singapore: implications for surveillance and response measures. Lancet 395, 1039-1046.
Qu, J., Wu, C., Li, X., Zhang, G., Jiang, Z., Li, X., Zhu, Q., and Liu, L. (2020). Profile of IgG and IgM antibodies against severe acute respiratory syndrome coronavirus 2 (SARS-CoV-2). Clinical infectious diseases: an official publication of the Infectious Diseases Society of America.
Richardson, S. I., Lambson, B. E., Crowley, A. R., Bashirova, A., Scheepers, C., Garrett, N., Abdool Karim, S., Mkhize, N. N., Carrington, M., Ackerman, M. E., et al. (2019). IgG3 enhances neutralization potency and Fc effector function of an HIV V2-specific broadly neutralizing antibody. PLoS pathogens 15, e1008064.
Seow, J., Graham, C., Merrick, B., Acors, S., Steel, K. J. A., Hemmings, O., O'Bryne, A., Kouphou, N., Pickering, S., Galao, R., et al. (2020). Longitudinal evaluation and decline of antibody responses in SARS-CoV-2 infection. medRxiv, 2020.2007.2009.20148429.
Singapore Statutes Online (2003). Infectious Diseases Act (https://sso.agc.gov.sg/Act/IDA1976; Sep. 9, 2020).
Taborda, C. P., Rivera, J., Zaragoza, O., and Casadevall, A. (2003). More is not necessarily better: prozone-like effects in passive immunization with IgG. J Immunol 170, 3621-3630.
Tai, W., He, L., Zhang, X., Pu, J., Voronin, D., Jiang, S., Zhou, Y., and Du, L. (2020). Characterization of the receptor-binding domain (RBD) of 2019 novel coronavirus: implication for development of RBD protein as a viral attachment inhibitor and vaccine. Cell Mol Immunol 17, 613-620.
US-FDA (2020). Coronavirus (COVID-19) Update: Daily Roundup Aug. 17, 2020 (https://www.fda.gov/news-events/press-announcements/coronavirus-covid-19-update-daily-roundup-august-17-2020: Aug. 17, 2020).
Walker, M. R., Eltahla, A. A., Mina, M. M., Li, H., Lloyd, A. R., and Bull, R. A. (2020). Envelope-Specific IgG3 and IgG1 Responses Are Associated with Clearance of Acute Hepatitis C Virus Infection. Viruses 12.
Wong, J. E. L., Leo, Y. S., and Tan, C. C. (2020). COVID-19 in Singapore-Current Experience: Critical Global Issues That Require Attention and Action. Jama.
Wu, F., Zhao, S., Yu, B., Chen, Y. M., Wang, W., Song, Z. G., Hu, Y., Tao, Z. W., Tian, J. H., Pei, Y. Y., et al. (2020). A new coronavirus associated with human respiratory disease in China. Nature 579, 265-269.
Zhang, B., Zhou, X., Zhu, C., Song, Y., Feng, F., Qiu, Y., Feng, J., Jia, Q., Song, Q., Zhu, B., et al. (2020). Immune Phenotyping Based on the Neutrophil-to-Lymphocyte Ratio and IgG Level Predicts Disease Severity and Outcome for Patients With COVID-19. Front Mol Biosci 7, 157.
Zhou, P., Yang, X. L., Wang, X. G., Hu, B., Zhang, L., Zhang, W., Si, H. R., Zhu, Y., Li, B., Huang, C. L., et al. (2020). A pneumonia outbreak associated with a new coronavirus of probable bat origin. Nature 579, 270-273.
Zollinger, W. D., and Mandrell, R. E. (1983). Importance of complement source in bactericidal activity of human antibody and murine monoclonal antibody to meningococcal group B polysaccharide. Infection and immunity 40, 257-264.
Zou, L., Ruan, F., Huang, M., Liang, L., Huang, H., Hong, Z., Yu, J., Kang, M., Song, Y., Xia, J., et al. (2020). SARS-CoV-2 Viral Load in Upper Respiratory Specimens of Infected Patients. The New England journal of medicine 382, 1177-1179.

### APPLICATIONS

Embodiments of the methods disclosed herein provide a fast, efficient and cheap way of detecting serological marker indicative of a coronavirus infection. Embodiments of the disclosed methods also seek to overcome the problems of providing a specific serological marker that enables the confirmation of negative or borderline results from other serological tests.

Advantageously, the methods as disclosed herein may independently confirm test for borderline subjects and fast determination of previous viral exposure.

Even more advantageously, the methods as disclosed herein may be based on the full-length S protein, which allows the capture of the full repertoire of specific antibodies against the S protein, especially conformational epitope of the S protein. In comparison, most serological tests are directed recombinant proteins (Spike or N proteins) on paper-based device or lateral flow/immunochromatographic assays and detect total IgG, IgM or IgA. In addition, other assays based on the S protein detect only antibodies against the receptor-binding domain (RBD) of the S protein, but not other domains of the S protein and/or conformational epitope of the S protein.

The methods as disclosed herein also have high sensitivity (100%) for specific IgG against the S protein as the assay is based on the full-length S protein, which allows the capture of the full repertoire of specific antibodies against the S protein. In comparison, assays based on the RBD of the S protein has 94% sensitivity.

The data as disclosed herein provides support that the methods as disclosed herein could be used as a screen to detect positive SARS-CoV2 cases.

The methods as disclosed herein have also been applied to detect IgM for plasma from patients at early stages of infection or in patients experiencing/having mild symptoms. In addition, the methods as disclosed herein have also been applied to detect functional IgG1 and IgG3, which are important in neutralizing virus.

Advantageously, the methods as disclosed herein may be a FACS-based assay that allows multiplexing. That is, different isotypes and/or IgG subclasses could potentially be detected and distinguished in a single test. By being able to distinguish the IgG, and IgM response, it might inform on the stage of infection of the patients. This could be particularly important for asymptomatic patients. Similarly, the methods as disclosed herein may be applied to detect and distinguish different IgG subclasses together in one test. In comparison, an ELISA-based assay can detect IgG or IgM separately, but not together.

Even more advantageously, the results generated by the methods as disclosed herein can be obtained in no more than about 1-2 hours.

The methods as described herein may be more sensitive than ELISA which can detect IgG but not in 100% of patients tested (unlike embodiments of the assay described herein).

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of identifying/characterizing coronavirus infection, optionally SARS-CoV-2 infection in a subject, the method comprising:
contacting a sample from the subject with an isolated host cell comprising a nucleic acid encoding for the spike protein (S protein) of the coronavirus; and
detecting a binding of an antibody or fragment thereof to the host cell.

2. (canceled)

3. The method of claim 1, wherein the detecting step comprises using a fluorescence detection instrument, optionally wherein the instrument is a flow cytometer to detect a binding of antibody or fragment thereof to the host cell.

4. The method of claim 1, the method further comprising contacting/incubating the sample with one or more detection or secondary antibody.

5. The method of claim 1, wherein the method is a diagnostic method.

6. The method of claim 1, the method further comprising performing a further assay such as a polymerase chain reaction (PCR)-based assay to detect/confirm coronavirus infection, optionally SARS-CoV-2 infection

7. The method of claim 1, wherein the method is an in-vitro or an ex-vivo method.

8.-17. (canceled)

18. The method of claim 1, wherein the method is a serological diagnostic method.

19. A method of identifying a subject having immunity for coronavirus infection, optionally SARS-CoV-2 infection, the method comprising:
contacting/incubating a sample from the subject with an isolated host cell comprising a nucleic acid encoding for the spike protein (S protein) of the coronavirus; and
detecting a binding of an antibody or fragment thereof to the host cell.

20. The method of claim 19, wherein the detecting comprises using a fluorescence detection instrument, optionally wherein the instrument is a flow cytometer to detect a binding of antibody or fragment thereof to the host cell.

21. The method of claim 19, further comprising contacting/incubating the sample with one or more detection or secondary antibody.

22. The method of claim 19, wherein the method is a diagnostic method.

23. The method of claim 19, wherein the method is a serological diagnostic method.

24. The method of claim 19, further comprising performing a further assay such as a polymerase chain reaction (PCR)-based assay to detect/confirm coronavirus infection, optionally SARS-CoV-2 infection.

25. The method of claim 19, wherein the method is an in-vitro or an ex-vivo method.

26. A product comprising:
a) a nucleic acid, optionally an isolated and/or recombinant nucleic acid, encoding for the spike protein (S protein) of severe acute respiratory syndrome (SARS)-like coronavirus (SARS-CoV-2), or
b) a viral vector, optionally a recombinant viral vector, comprising a nucleic acid, optionally an isolated and/or recombinant nucleic acid, encoding for the spike protein (S protein) of severe acute respiratory syndrome (SARS)-like coronavirus (SARS-CoV-2), or
c) a host cell, optionally an isolated host cell, comprising a nucleic acid, optionally an isolated and/or recombinant nucleic acid, encoding for the spike protein (S protein) of severe acute respiratory syndrome (SARS)-like coronavirus (SARS-CoV-2), optionally wherein the host cell is transduced with a viral vector, optionally a recombinant viral vector, comprising a nucleic acid, optionally an isolated and/or recombinant nucleic acid, encoding for the spike protein (S protein) of severe acute respiratory syndrome (SARS)-like coronavirus (SARS-CoV-2).

27. The product of claim 26, wherein the host cell expresses the S protein, optionally a full-length S-protein.

28. The product of claim 26, wherein the S protein comprises a full-length S protein.

29. The product of claim 26, wherein the S protein comprises SEQ ID NO: 1, SEQ ID NO: 3, SEQ ID NO: 4, SEQ ID NO: 5, or a sequence sharing at least about 75% sequence identity with SEQ ID NO: 1, SEQ ID NO: 3, SEQ ID NO: 4, or SEQ ID NO: 5, or
wherein the nucleic acid comprises SEQ ID NO: 2, SEQ ID NO: 6, SEQ ID NO: 7, SEQ ID NO: 8, or a sequence sharing at least about 75% sequence identity with SEQ ID NO: 2, SEQ ID NO: 6, SEQ ID NO: 7, or SEQ ID NO: 8, or
wherein the nucleic acid comprises SEQ ID NO: 2, or a sequence sharing at least about 75% sequence identity with SEQ ID NO: 2, or
wherein the S protein/the full-length S protein comprises SEQ ID NO: 1 or a sequence sharing at least about 75% sequence identity with SEQ ID NO: 1.

30. The product of claim 26, wherein the host cell is bound to an antigen binding protein or a fragment thereof, optionally an antibody, further optionally an antibody against SARS-CoV-2.
